(19) United States
(12) Patent Application Publication
(10) Pub. No.: US 2025/0082909 A1
(43) Pub. Date: Mar. 13, 2025

(54) CATHETERS WITH GUIDEWIRE ADAPTORS AND RELATED METHODS

(71) Applicant: B. BRAUN MELSUNGEN AG, Melsungen (DE)

(72) Inventors: Teng Lip KHOO, Penang (MY); Lilian Zhi Ling LEW, Penang (MY); Mohd Zairizal BIN ZAKARIA, Penang (MY); Chee Mun PHANG, Penang (MY); Keng Seang TAN, Penang (MY); Kavintharan N/A, Penang (MY); Dominik JAROS, Melsungen (DE); Andre WEISS, Melsungen (DE)

(73) Assignee: B. BRAUN MELSUNGEN AG, Melsungen (DE)

(21) Appl. No.: 18/696,096

(22) PCT Filed: Sep. 29, 2022
(86) PCT No.: PCT/EP2022/077142

§ 371 Date: Mar. 27, 2024

Related U.S. Application Data
(60) Provisional application No. 63/250,142, filed on Sep. 29, 2021.

(51) Int. Cl. A61M 25/09

(52) U.S. Cl. CPC A61M 25/09041

(57) ABSTRACT

A catheter assembly having a catheter hub and a needle hub having an interior, an adaptor having a tip is disposed in the interior of the needle hub, and a guidewire dispenser having a connection tip located in a bore of the adaptor. A guidewire can extend through the dispenser and into the adaptor for use to guide a catheter tube. The adaptor can have a valve to restrict or limit blood flow in the proximal direction. Gas channels can be provided on an exterior of the adaptor to facilitate air venting and blood flashback.

### CROSS-REFERENCE TO RELATED APPLICATION

This is a US National Phase application of PCT Application No. PCT/EP2022/077142, filed Sep. 29, 2022, which claims priority to U.S. Provisional Application No. 63/250,142, filed Sep. 29, 2021, the contents of each of which are expressly incorporated herein by reference.

### FIELD OF ART

The disclosed invention generally relates to needle devices and intravenous (IV) infusion devices, including IV catheters. Specific aspects of IV catheter assemblies having an adaptor for use with a guidewire dispenser are disclosed.

### BACKGROUND

IV catheters are commonly used for a variety of infusion therapies, including infusing fluids into a patient, withdrawing blood from a patient, or monitoring various parameters of the patient's vascular system. Catheters are typically connected to a catheter adaptor that accommodates the attachment of IV tubing to the catheter. Blood control catheters include an internal blood control valve that is opened by the insertion of a male Luer or other objects into a proximal end of the catheter adaptor. Non-limiting examples of blood control valves are disclosed in United States Patent Application Publication No. 2011/0046570, filed Aug. 20, 2009, titled “Systems and Methods for Providing a Flushable Catheter Assembly.” Following placement of the catheter into the vasculature of a patient, an IV fluid source can be connected to the catheter adaptor or catheter hub, opening the blood control valve. Thus connected, fluid from the IV source can begin flow into a patient through the catheter.

As is well known in the art, typical blood pressure is 10 to 20 centimeters of water. Infusion bags are usually placed about 100 cm above the patient's heart to direct flow into the patient. At roughly that height, the pressure exerted by the fluid from the infusion bag is much greater than the blood pressure of the patient and therefore can flow into the patient.

For patients with difficult to access veins, extended dwell catheters can be used to aid in Difficult Intravenous Access (DIVA). An extended dwell catheter may be considered a peripherally inserted catheter. The extended dwell catheter can be configured for insertion in a larger vein than those used for standard I.V. therapy and comparatively can have a longer catheter length than typical peripheral access catheters. For DIVA patients, a physician can use visualization equipment to aid in identification of deep veins for catheter access. In that case, an extended dwell catheter can provide a longer length and a more flexible catheter for insertion into the patient. With the addition of a guidewire, this can help to reduce the chances of the catheter becoming kinked.

### SUMMARY

Aspects of the invention broadly cover catheter assemblies. In an example, a catheter assembly comprises a catheter hub and a needle hub having an interior with the needle projecting through the catheter tube. An adaptor having a tip can be disposed in the interior of the needle hub and a guidewire dispenser having a connection tip can be located in the bore of the adaptor. A guidewire can extend through the dispenser and into the adaptor for use to guide a catheter tube. The adaptor can have a valve to restrict or limit blood flow in the proximal direction or can have a guidewire passage with no or with tight clearance to restrict or limit blood flow in the proximal direction. In some examples, as discussed below, the guidewire dispenser and guidewire can be used with the adaptor.

Aspects of the invention further include a catheter assembly comprising: a catheter tube attached to a catheter hub and a needle attached to a needle hub, wherein the needle is located in a lumen of the catheter tube and a needle tip of the needle projects out a distal opening of the catheter tube; an adaptor having a body comprising a base, a tip, and a bore extending through the body, the base having a base bore section and the tip having tip bore section with a frustoconical shape, the tip located in a proximal opening of the needle hub; and a guidewire dispenser having a frame with a plurality of branches, a connection tip at a first branch of the plurality of branches located inside the base bore section of the adaptor; and wherein a guidewire extends through a second branch of the plurality of branches and in through the connection tip and into the bore of the adaptor. However, the tip can have other bore shapes and still encompasses aspects of the invention.

A housing can be use with the catheter assembly. The housing can comprise a first frame attached to a second frame and the two frames defining an interior space, said housing having a distal opening and a proximal opening and the catheter hub is located at least in part in the interior space of the housing. The housing can have the catheter assembly located therein and a guidewire adapter can attach to the needle hub of the catheter assembly. In some examples, the catheter assembly can be used without the housing.

A duct having a first end can be included with the guidewire dispenser. The first end of the duct can attach to a socket at the second branch and wherein the guidewire can extend at least partially into the duct.

The duct can have a second end attached to a socket of a third branch of the plurality of branches of the guidewire dispenser.

The duct and the frame of the guidewire dispenser define an enclosed loop. The enclosed loop can be generally round. The duct can be a length of tubing.

A valve can be located in the bore of the adaptor. The valve can have a valve disc with at least one slit and at least two valve flaps. In some examples, there can be three slits defining three valve flaps.

The valve can comprise a valve disc and a skirt extending therefrom. The valve disc can have different thickness sections.

A needle guard can be located in an interior of the catheter hub. The needle guard can have a proximal wall and at least one arm extending distally of the proximal wall. The arm can have a surface located to a side of the needle in a ready to use position and movable distal of the needle tip in a protective position.

The needle can have a crimp for engaging the needle guard during retraction of the needle. The needle can have a lumen. The guidewire can abut the needle at the crimp to delimit distal travel of the guidewire.

The guidewire can have at least two guidewire sections with at least two different diameters.

The guidewire can have a distal guidewire section of a first diameter and a proximal guidewire section of a second diameter, and wherein the second diameter is larger than the first diameter.

The guidewire can comprise a shoulder or a gradual taper located at a point between the distal guidewire section and the proximal guidewire section.

At least one of the shoulders, the gradual taper, and the second diameter can engage with the needle at the crimp.

A location of the shoulder or the gradual taper on the guidewire defines a length of the distal guidewire section that extends out the needle tip when the guidewire engages the needle at the crimp.

The distal guidewire section can pass through the needle at the crimp and can extend out an opening at the needle tip.

The guidewire can have a marker or an indicator to indicate how far the distal guidewire section has extended distally beyond the needle tip.

The marker or indicator on the guidewire can indicate at least one of when a first end of the distal guidewire section reaches the needle tip, passes through the needle tip, and advances fully out of the distal tip.

The guidewire can have a plurality of markers or guidewires to indicate when a first end of the distal guidewire section reaches the needle tip, passes through the needle tip, and advances fully out of the distal tip.

The guidewire can have a code, the guidewire dispenser can have a code, or both the guidewire and the guidewire dispenser can have codes for indicating a guidewire gauge size.

The code or codes can include a color, an alpha code, a numeric code, an alpha numeric code, or combinations thereof.

The tip of the adaptor can comprise a cannula channel having an inside diameter and a guidewire passage having an inside diameter, and wherein the diameter of the cannula channel is larger than the diameter of the guidewire passage.

The cannula channel is located distal of the guidewire passage.

The base of the adaptor can have a body with an exterior having plurality of slats.

Two or more flanges can extend from two or more of the slats on the base of the adaptor.

One of male detent and female detent can be formed with each of the two or more flanges.

The body at the tip of the adaptor can have a cannula channel having an interior surface defining a non-circular cross-sectional opening.

The proximal end of the needle can contact the interior surface at the cannula channel when the catheter assembly is in a ready to use position.

The non-circular cross-sectional opening of the cannula channel can have a plurality of straight sides and a plurality of arcuate connecting sections.

The needle can contact the straight sides of the cannula channel.

Bypass gaps can be formed between the plurality of arcuate connecting sections and the needle.

The body at the tip of the adaptor can have a plurality of bore sections of different internal diameters.

The plurality of branches of the guidewire dispenser can comprise at least three branches, which include a first branch, a second branch, and a third branch.

A tubing having a first end, a second end, and a lumen and wherein the guidewire can be disposed, at least in part, in the lumen of the tubing.

The first end of the tubing can be located in a socket at the second branch and the second end of the tubing can either be located in a socket at the third branch or be remote from the third branch.

An intermediate retaining clip can grip different sections of the tubing. The intermediate retaining clip can have two slots. Optionally, the intermediate retaining clip can be omitted.

A valve can be located in the catheter hub.

A valve opener can be located in the catheter hub.

The adaptor embodiments described herein can be separately packaged or provided from the catheter assembly. After successfully accessing a vessel, the vent plug can be removed from the needle hub, the adaptor is then connected to the needle hub, and the guidewire dispenser can then connect to the adaptor.

In some examples, the adaptor can be pre-packaged with the catheter assembly, such as mounted to the catheter hub. For example, the adaptor can have a valve located therein for restricting flow out the proximal end of the adaptor so that the catheter assembly can be used to gain access to a vessel while the adaptor is connected to the needle hub.

A further aspect of the invention includes an adaptor for use to guide a guidewire through a needle lumen comprising: a body comprising a base, a tip, and a bore extending through the body; the base having a base bore section and the tip having tip bore section with a frustoconical shape; at least one of a cannula channel at the tip having a plurality of generally straight sides with two adjacent plurality of straight sides connected to one another by an arcuate connecting portion and sized to receive a needle shaft and a valve comprising a wall having at least one slit positioned in the base bore of the base; wherein the base comprises a proximal opening sized and shaped for receiving a male tip of a guidewire dispenser and plurality of first engagement structures for engaging a corresponding plurality of second engagement structures on the male tip of the guidewire dispenser.

The base of the adaptor can have a non-circular cross-section having a plurality of slats or sidewalls.

Each one of the plurality of first engagement structures on the adaptor can extend from one of the plurality of slats or sidewalls. The first engagement structures can engage second engagement structures on the guidewire dispenser.

The cannula channel can have a first inside cross-sectional dimension and a guidewire passage can have a second inside cross-sectional dimension located proximally of the cannula channel; and wherein the first inside cross-sectional dimension can be greater than the second inside cross-sectional dimension.

The cannula channel can have three generally straight sides and three arcuate connecting portions.

The valve can have a skirt extending from the wall, and the wall can have at least one slit defining at least two flaps.

The wall of the valve can have a first portion with a first thickness and a second portion with a second thickness and wherein the first thickness is greater than the second thickness, measured orthogonal to a medial plane passing through the wall.

The valve can be located between two internal shoulders of the base.

Yet another feature of the invention is an adaptor for use to guide a guidewire through a needle lumen comprising: a body comprising a base, a tip, and a bore extending through the body; the base having a base bore section and the tip having tip bore section; a plurality of gas channels disposed on an exterior of the tip; wherein the base comprises a proximal opening sized and shaped for receiving a male tip of a guidewire dispenser; and wherein each of the gas channel comprises a first channel end located proximate a shoulder between the tip and the base.

The adaptor can further comprise a protective cap having an end disposed over the tip of the adaptor and a connection tip of a guidewire dispenser disposed in the base bore section, and

wherein a duct can comprise a lumen attached to the guidewire dispenser.

A further aspect of the invention includes a guidewire dispenser comprising: a frame with a plurality of branches, including a first branch and a second branch; a connection tip at the first branch, said connection tip comprising a lumen and a tapered nose section; a guide bracket at the second branch and a lumen formed through guide bracket, said guide bracket comprising a socket; a tubing having a first free end and a second free end, said first free end located in the socket and said second free end held by a retaining clip or by socket located on a third branch; a guidewire having a length with a first free end and a second free end, said guidewire located in said tubing and extending through the socket at the second branch and into the lumen of the connection tip; and wherein the guidewire is exposed between the first branch and the second branch.

The third branch of the guidewire dispenser can have an enclosed wall at an end of the socket. The third branch can have not pass through. Alternatively, the third branch can have a C-channel.

The second branch can be located higher, elevation-wise, than the first branch.

The tubing or duct and the frame can form an enclosed loop.

The guidewire can have a first section with a first diameter and a second section with a second diameter larger than the first diameter.

The frame of the guidewire dispenser can have a Y-shape configuration.

Two or more retaining clips can be used to retrain different sections of the tubing or duct.

A still further aspect of the invention is a catheter assembly comprising: a catheter tube attached to a catheter hub and a needle having a needle tip attached to a needle hub, wherein the needle projects through the catheter tube and the needle tip disposed distally of a distal opening of the catheter tube; an adaptor having a body with an exterior surface and an interior surface defining a bore, the body further having a base with a plurality of slats connected to one another along respective edges and a tip having a frustoconical section having a guidewire passage and a cannula channel distal of the guidewire passage, said tip located in a proximal opening of the needle hub; a guidewire dispenser having a tip disposed in the bore of the adaptor and a guidewire having a first end disposed in the guidewire passage; and wherein a proximal end of the needle is disposed in the cannula channel of the adaptor. However, the tip can have other bore shapes and still encompasses aspects of the invention.

A yet further aspect of the invention is a catheter assembly comprising: a catheter tube attached to a catheter hub and a needle having a needle tip attached to a needle hub, wherein the needle projects through the catheter tube and the needle tip disposed distally of a distal opening of the catheter tube; an adaptor having a body with an exterior surface and an interior surface defining a bore, the body further having a base and a tip. A shoulder can be located between the base and the tip. Optionally, the smooth tapered transition can be located between the base and the tip. A bore can extend through the adaptor, and through the base to define a valve chamber. A valve comprising at least one slit and two flaps can be located in the valve chamber of the adapter. The tip can have a frustoconical section having a guidewire passage, said tip located in a proximal opening of the needle hub. A guidewire dispenser having a tip can be disposed in the bore of the adaptor and a guidewire having a first end can be disposed in the bore of the adaptor. A guidewire passage and a cannula channel can be located at a distal end of the tip of the adapter.

Yet another embodiment of the invention can include a catheter assembly comprising: a catheter tube attached to a catheter hub and a needle attached to a needle hub, wherein the needle is located in a lumen of the catheter tube and a needle tip of the needle projects out a distal opening of the catheter tube; an adaptor having a body comprising a base, a tip, and a bore extending through the body, the base having a base bore section and the tip having tip bore section, the tip located in a proximal opening of the needle hub; and a guidewire dispenser having a frame with a plurality of branches, a connection tip at a first branch of the plurality of branches located inside the base bore section of the adaptor; and wherein a guidewire extends through a second branch of the plurality of branches and in through the connection tip at the first branch.

The present invention still further includes a method for assembly a catheter assembly comprising: providing a catheter hub with a catheter tube extending out a distal end of the catheter hub, said catheter hub having a body with an interior cavity and said catheter tube comprising a lumen and a distal opening; providing a needle hub with a needle having a shaft with a lumen and a needle tip, said needle hub comprising a body with an interior cavity and said needle projecting through the lumen of the catheter tube with said needle tip projecting distally of the distal opening of the catheter tube; providing an adaptor having a body with an exterior surface and an interior surface defining a bore, the body further having a base having a base bore and a tip, said tip of said adaptor located in the interior cavity at a proximal opening of the needle hub; and providing a guidewire dispenser, said guidewire dispenser having a connection tip disposed in the base bore of the adaptor and a guidewire extending across an advancement platform of the guidewire dispenser.

Yet another aspect of the invention is an adaptor for use to guide a guidewire through a needle lumen comprising: a body comprising a base, a tip, and a bore extending through the body; the base having a base bore section and the tip having tip bore section; a plurality of gas channels disposed on an exterior of the tip; wherein the base comprises a proximal opening sized and shaped for receiving a male tip of a guidewire dispenser; and wherein each of the gas channel comprises a first channel end located proximate a shoulder between the tip and the base.

A plurality of elongated ribs can be provided on the first and second tip sections of the tip. Each rib can have a length extending in the axial direction of the adaptor, a width measured orthogonally to the length, and height measured from the exterior surface of the tip to the top surface of the rib.

In an example, the collective top surfaces of the plurality of ribs define an effective diameter. In some examples, the height of each rib can vary from the distal end of the tip to the second end of the transition section. Thus, the effective diameter of the tip at the third tip section can vary due to the variable heights of the ribs.

When incorporated, the effective diameter of the tip at the third tip section can create a taper, or no taper, to enable the tip to fit inside the bore of the needle hub and match the taper contour of the bore of the needle hub. The effective diameter of the tip at the second tip section can similarly be selected to fit inside the bore of the needle hub and match the taper contour of the bore.

Preferably, the effective diameter defined by the plurality of ribs and the exterior of the first tip section form a continuous outer diameter profile that can fittingly couple to a bore of a needle hub when inserting the tip into the proximal opening of the needle hub. The effective diameter at the distal end of the tip is preferably smaller than the rest of the effective diameter so as to create a reduced tip end to facilitate insertion of the tip into the needle hub.

A ravine is defined between two adjacent elongated ribs. Each ravine has a width and a length. Relative to the effective diameter of the tip, each ravine also has a depth measured relative to the top surfaces of the adjacent ribs. Thus, the tip of the present embodiment comprises a plurality of ribs and a plurality of ravines. In an example, four elongated ribs defining four ravines are provided on the tip of the present adaptor. In some examples, there can be fewer than four ribs or more than four ribs, such as three ribs, five ribs, or six ribs, defining three ravines, five ravines, or six ravines, respectively.

The ribs are preferably spaced apart around the periphery of the tip to define four generally same sized ravines. However, the tip of the present adaptor can still be inserted into the proximal opening of a needle hub and couple to the needle hub when unequally spaced ribs and ravines of different sizes are incorporated. Gas channels can be provided on the tip. The gas channels can be sized and shaped to permit gas, such as air, to travel inside the channels to escape from the interior space of the needle hub but not large enough to permit viscous fluids, such as blood, to freely flow therein.

In an example, each gas channel is aligned axially relative to the longitudinal axis of the adaptor. Each gas channel can have a first channel end located at about the shoulder, such as distal of the shoulder, and a second channel end located distal of the first end of the second tip section. In an example, the second channel end can originate or terminate, depending on the viewing perspective, somewhere on the third tip section.

The second channel end of each gas channel is preferably located somewhere between the first end and the second end of the second tip section. The first and second channel ends can be understood to form two ends of each gas channel. Less preferably, different second channel ends of a plurality of gas channels are located along different sections of the tip, such as some being located at the second tip section while others are located at the third tip section.

As discussed above, the first channel end of each gas channel can originate or terminate at the shoulder between the tip and the base. In some examples, the first channel ends of some or all of the gas channels can be located distal of the shoulder, somewhere along the first tip section at the gap when the adaptor is coupled to the needle hub and the guidewire dispenser.

The gas channels allow air to escape by entering through the second channel end and flowing in the proximal direction in the respective gas channel. Without the gas channels, the first tip section can form a continuous taper fit with the interior of the needle hub. Thus, air trapped inside needle hub during blood flashback may not have sufficient vent openings in the system for venting. The gas channels incorporated with the present adaptor provides air passages through an otherwise continuous seal between the interior of the needle hub and the exterior of the tip, such as the exterior of the first tip section.

When the present adaptor with the gas channels is used as part of a PG unit with a catheter assembly, initial puncture of a vein will cause blood to enter the needle lumen and into the interior space of the needle hub, known as primary blood flashback. By incorporating the plurality of gas channels, air can be exhausted out of the needle hub interior space as blood travels into the flashback chamber of the needle hub, thereby promoting blood flashback flow and minimizing vapor lock when no air or little air is allowed to escape. In an example, four or more gas channels can be incorporated with the tip. Preferably six evenly spaced gas channels are incorporated with the tip. More preferably, eight gas channels are incorporated with the tip.

The present adaptor with gas channels may be used in a packaging as a guiding unit, with a protective sleeve and a guidewire dispenser.

The invention further includes a guidewire dispenser comprising: a frame with a plurality of branches, including a first branch and a second branch; a connection tip at the first branch, said connection tip comprising a lumen; a guide bracket at the second branch and a lumen formed through guide bracket, said guide bracket comprising a socket; a tubing having a first free end and a second free end, said first free end located in the socket and said second free end held by a retaining clip or by socket located on a third branch; a guidewire having a length with a first free end and a second free end, said guidewire located in said tubing and extending through the socket at the second branch and into the lumen of the connection tip; and wherein the guidewire is exposed between the first branch and the second branch.

A still yet further aspect of the invention is a method for assembly a catheter assembly comprising: providing a catheter hub with a catheter tube extending out a distal end of the catheter hub, said catheter hub having a body with an interior cavity and said catheter tube comprising a lumen and a distal opening; providing a needle hub with a needle having a shaft with a lumen and a distal tip, said needle hub comprising a body with an interior cavity and said shaft projecting through the lumen of the catheter tube with said distal tip projecting distally of the distal opening of the catheter tube; providing an adaptor having a body with an exterior surface and an interior surface defining a bore, the body further having a base and a tip having a frustoconical section. A bore can extend through the base and the tip can have a guidewire passage. The bore at the base and the guidewire passage can be in fluid communication. The tip of the adaptor can be located in the interior cavity at a proximal opening of the needle hub. The method can further comprise the step of providing a guidewire dispenser, said guidewire dispenser having a tip disposed in the base bore of the adaptor and a guidewire having a first end disposed in the base bore of the adaptor.

The distal tip of the needle can have a needle bevel. As the needle can be hollow, the bevel tip can have an opening.

A method of using a catheter assembly with a guidewire comprising: puncturing a vein with a needle tip of a needle, which has a needle crimp; advancing a guidewire through an adaptor connected to a needle hub having the needle attached thereto; and further advancing the guidewire so that a distal guidewire section extends out the needle tip and the guidewire engages the needle at needle crimp.

The guidewire can be advanced through the needle without a guidewire dispenser.

The method can include dispensing the guidewire from a guidewire dispenser, and wherein the guidewire dispenser can have a tip connected to a base of the adaptor.

In an example, the catheter assembly can be practiced as extended dwell catheter assembly. The extended dwell catheter assembly can comprise a housing having a receiving space or interior space having a catheter assembly located therein.

The catheter assembly can have a vent plug engaging the proximal opening of the needle hub. In alternative embodiments, the vent plug can be replaced with an adaptor and a guidewire dispenser, which is coupled to or engaged to the adaptor. The catheter assembly with the adaptor and guidewire dispenser may optionally be used with the housing or may be used without the housing. In other examples, a guidewire can project through the adaptor without having to use the guidewire dispenser to connect to the adaptor.

In some examples, a crimp can be incorporated with the needle for engaging an opening on the needle guard, when withdrawing the needle following successful venipuncture. When the crimp is used, the crimp can define a relatively smaller internal opening in the needle lumen than the nominal inside diameter of the needle lumen. The relatively smaller internal opening at the crimp can be used as a limiter, physical barrier, or limiting means to limit distal advancement of the guidewire. For example, the guidewire can include a bead or an enlarged section at a predetermined point along the guidewire between the two ends, i.e., between a first end at the distal end and a second end at the proximal end.

When the bead or the enlarged section on the guidewire passes through the needle, after the distal end of the guidewire advances out through the needle tip, the bead or the enlarged section is physically restricted from passing distally through the relatively smaller internal opening at the crimp, thereby stopping any further advancement of the first end of the guidewire out of the needle tip. The location of the bead or the enlarged section relative to the first end of the guidewire can be selected to control the length of the first end that travels out the needle tip, and therefore enters the vasculature of the patient when the guidewire is in use.

In some examples, the guidewire is provided with at least two different guidewire sections with two different outside diameter dimensions. The distal guidewire section can have a first OD and the proximal guidewire section can have a second OD and wherein the second OD is larger than the first OD. The first OD can be sufficiently small to pass through the needle lumen at the crimp but not the second OD. The length of the distal guidewire section can be selected to represent the length of the guidewire that will pass through the patient's vasculature or vein when the guidewire is stopped at the crimp. A shoulder can be provided at the interface between the two guidewire sections. In other examples, a gradual transition can be provided between the two guidewire sections. The shoulder, the gradual transition, or the second OD of the guidewire can be sized to abut the crimp section of the needle when dispensing the guidewire out through the needle tip. Thus, the assembly can be provided with a safe stop to stop further advancement of the guidewire into the vein. In some examples, rather than a crimp section, the gradual transition, or the second OD of the guidewire can be sized to abut a reduced needle section at the needle notch to stop distal advancement of the guidewire.

The catheter assembly has a catheter tube and a needle located inside the lumen of the catheter tube and both extending out a distal end of the housing. A needle tip of the needle can extend out a distal opening of the catheter tube. The catheter tube can attach to a catheter hub and the needle can attach to a needle hub. The needle hub can extend out a proximal end of the housing of the extended dwell catheter assembly. The needle hub can include a vent plug, which can engage the proximal opening of the needle hub, such as in a Luer fit. For a relatively longer length catheter tube and needle, an adaptor and a guidewire dispenser can replace the vent plug.

In an example, a catheter assembly comprises a first hub or catheter hub having an over-the-needle tube or catheter tube attached thereto and a second hub or needle hub having a needle attached thereto. The catheter tube can have a lumen or bore for receiving the needle and has a distal opening or distal end opening and the needle tip of the needle extends distally of the distal opening in a ready to use position, in which the assembly is ready for venipuncture or for accessing the vasculature of a patient. The catheter assembly can also be called a needle assembly.

The needle can include a change in profile located proximally of the needle tip for use with a needle guard. The needle tip can extend distally of the distal end opening of the tube or tubing in the ready to use position with the change in profile, if incorporated for use with a needle guard, located proximally of the distal end opening. The change in profile can be a crimp, a bump, or a material build-up having different profile than other diameter sections of the needle shaft. As used herein, the term proximal is understood to mean an end or side closer to the practitioner and the term distal is the opposite end or side.

The change in profile can be used to interact with a needle guard during retraction of the needle following successful venipuncture, as further discussed below. A vent plug is disposed at the proximal open end of the second hub or needle hub. The vent can have a vent filter at a proximal end thereof, which is conventional. The catheter tube can attach to the first hub or catheter hub by a ferrule or bushing.

A needle guard can be provided in the interior cavity of the first hub or catheter hub for covering the needle tip in the protective position. When incorporated, the needle guard can be one of the needle guards disclosed in U.S. Pat. No. 6,616,630, the contents of which are expressly incorporated herein by reference.

In some examples, the needle guard can be omitted. If so, the change in profile on the needle can also be omitted. However, where the change in profile is used as a stopping mechanism to stop distal advancement of a guidewire, the change in profile on the needle can be utilized even if the needle guard is omitted. In still other examples, a needle guard can be included without also including a change in profile on the needle. In yet other examples, the needle guard can be located in a third housing between the first hub and the second hub. An exemplary needle guard located in a third housing is disclosed in U.S. Pat. No. 8,597,249, the contents of which are expressly incorporated herein by reference. In still other examples, the needle guard can include multiple arms or fingers extending in both a first direction and an opposite second direction as disclosed in U.S. Pat. No. 9,387,307, the contents of which are expressly incorporated herein by reference. For example, the needle guard can include a first protector body surrounded by a second protector body.

In an example, the needle guard can be made from a metal material and can have a resilient portion that can generate a biasing force. The needle guard can comprise a wall surface that is positioned to a side of the needle and is movable distal of the needle tip to guard the needle tip from accidental contact therewith. The wall surface can be a needle trap and can move directly in front or distal of the point of the needle tip.

The needle guard can include a proximal wall and two arms extending distally of the proximal wall. The two arms can intersect one another in the ready to use position when viewed from a side and in the protective position in which the needle guard covers the needle tip, again as viewed from the side. In some examples, the two arms of the needle guard can extend in a distal direction without intersecting one another. Two distal walls, one on each arm, can be incorporated to block the needle tip.

The two distal walls can be biased outwardly by the needle in the ready to use position and disposed distally of an internal change in profile inside the first hub in the ready to use position. The internal change in profile can be a reduced diameter section located next to an inside diameter section of a larger dimension. The elbows on the two arms of the needle guard can have a dimension that is larger than the inside diameter at the internal change in profile to restrict proximal movement of the needle guard from the catheter hub until the two arms are no longer biased outwardly or until the dimension at the two elbows are reduced.

Each distal wall of each arm can include a curved lip to facilitate relative movement between the needle guard and the needle. When in the ready to use position, the dimension measured between the two joints or elbows, at the intersection between each arm and its respective distal wall, is larger than the inside diameter of the internal change in profile, which prevents the needle guard from moving proximally thereof due to the relative dimensions.

Once the needle tip moves proximally of the two distal walls, the two arms are allowed to move radially inward and one or both distal walls close over the needle tip to block the needle tip in a protective position. At this point, the dimension between the two joints or elbows decreases and is smaller than the internal dimension of the internal change in profile, which then allows the needle guard to move proximally and be removed from the catheter hub with the needle. In alternative embodiments, the dimension between the two joints can be equal to or slightly larger than the internal dimension of the internal change in profile and the needle can still move proximally by flexing or squeezing the two joints to get through the internal change in profile.

The first hub can have a proximal opening having a nose section of the second hub disposed therein. The proximal opening of the first hub has a female Luer for receiving a male Luer tip, such as a syringe, an IV tubing connector, a Luer extension set, etc. External threads can be provided on the exterior surface at the proximal end for threaded engagement with a threaded collar of a male threaded Luer tip.

A pair of stabilizing wings can extend radially of the first hub to facilitate securement or anchoring of the first hub to a patient following successful venipuncture. Optionally the wings can be omitted. The first hub can embody a standard IV catheter hub without an injection port. In other examples, the first hub can include an injection port or an integrated extension set. If the wings are not present on the first hub or catheter hub, then the bottom of the first hub can have a flattened surface angled at between 2 to 15 degrees from the through axis of the first hub.

The first hub can be removably secured to the second hub by receiving the nose section of the second hub in the proximal opening thereof. A flange or extension can be provided on the second hub and together with the nose section define a gap having part of the first hub located therein.

Optionally the flange or extension can be omitted, and a stub or other surface mating features provided. The stub can be the male part of a key fitting in a gap through or partially into the Luer threads to prohibit rotation of the second hub relative to the first hub. The proximal facing end surface of the first hub can abut the distally facing surface of the step in the outside surface of the second hub which is at the proximal end of the nose section, which is inside the first hub. This abutment can set the amount of protrusion of the needle tip out of the catheter tubing. The stub can be an optional structural feature.

The second hub can have a body having an optional interior cavity having the proximal end of the needle projecting there-into. The interior cavity can function as a primary blood flashback chamber. The proximal opening of the body can have a female Luer for receiving the vent plug, a syringe tip, or an adaptor therein and the exterior can have threads or be without threads. In an example, the exterior of the body of the second hub is generally cylindrical and without threads. The first and second hubs, or catheter hub and needle hub, respectively, can be made from plastic materials, such as by plastic injection.

A push tab can be provided with the first hub or catheter hub. The push tab can be located on an exterior surface of the first hub at a location distal of the exterior threads so as to avoid interfering with the exterior threads. For example, the push tab should be positioned sufficiently distally of the exterior threads so that when a threaded male connector, such as a syringe type with a threaded collar, is connected to the threads of the first hub, the push tab does not interfere with the connection. In some examples, the push tab should be positioned sufficiently distally of the exterior threads so that when the push tab is folded over, the push tab does not interfere with the connection.

In some examples, the catheter assembly can incorporate a valve and a valve opener. For example, a valve and a valve opener can both be located within the catheter hub. In use, the valve opener can be pushed distally into the valve to open the one or more slits formed with the valve to open a fluid passage through the valve for fluid flow. An exemplary catheter assembly with a valve and a valve opener is disclosed in U.S. Pat. No. 8,333,735, the contents of which are expressly incorporated herein by reference.

The valve opener can have a nose section having a bore and a plunger section located proximally of the nose section. The nose section can be pushed into the valve to open the one or more slits of the valve, such as three slits defining three flaps, to open the valve for fluid flow. The plunger section of the valve opener can comprise two or more plunger components having a gap therebetween. The gap can allow fluid to flow through the plunger components and through the bore in the nose section.

In an example, the housing for use in an extended dwell catheter assembly of the present invention can comprise a left frame or first frame and a second frame or right frame attached to one another. Each frame can comprise a nose portion, a body portion, and a base portion. The two nose portions can join to form a nose end, the two body portions can join to form a body, and the two base portions can join to form a base end.

In an example, the nose end or nose portion is provided with a nose tip having an elongated cylindrical like structure having a bore for accommodating the catheter tube and needle, which is located within the catheter tube. The nose end or nose portion can further include a nose base, which has an enlarged portion formed with the tip, and a tapered distal portion. A plurality of support ribs or fins can be provided at the intersection between the tip and the nose base. In other examples, the nose end can be a spherical or rounded dome having an opening to accommodate the catheter tube and needle.

The body of the housing can have an interior space for accommodating a catheter assembly, which can be the catheter assembly described elsewhere herein. A body opening or first opening can open into the interior space. The body opening can extend from the nose end to the base end.

In an example, a second opening opposing the first opening can be provided with the body so that the interior space can be accessed from the two body openings. Thus, the body therefore can have a through passage defined by the two body openings. In other examples, the body only has a single opening leading to the interior space as shown with the opposing side being solid or is enclosed by wall surfaces of the first frame and the second frame. Optionally the enclosed side can have small windows or openings for access or venting but not a full opening, like that of body opening.

The base can extend from the body and the base can have a plurality of sides. In an example, there can be four sides defining an interior space, which can open to the interior space of the body. In other examples, the base can embody a generally elongated cylinder or a non-circular cylinder. For discussion purposes, the sides can include a top side, a bottom side, a left side, and a right side. The sides may also be understood as the first side, the second side, the third side, and the fourth side. These specific nomenclatures are given so as to enable calling out a particular side relative to other sides only but are not limiting unless the context indicates otherwise.

The base can have an open proximal end to allow the needle hub to extend proximally thereof, proximal of an end surface, in the ready to use position. As shown, the first side and the second side of the base end can each have an opening to allow viewing and accessing the interior space of the base end, such as to facilitate or assist assembling of the catheter assembly therein, as further discussed below.

The top side of the base end can be provided with a top lever and the bottom side can be provided with a bottom lever. When a user simultaneously applies a compressive force and a distally directed force on the top lever and a compressive force and a proximally directed force on the bottom lever, the user can cause the left housing frame and the right housing frame to move relative to one another and the detents on the first frame and second frame to separate so that the housing can then separate into separate housing components, i.e., the left frame and the right frame. In other words, by manipulating the top lever and the bottom lever, the user can cause the housing to separate into two or more housing components along the separation line.

The separation of the housing into housing components can then expose the catheter assembly to separate from the housing following successful venipuncture so that the catheter hub can then be secured to an IV line and secured to the patient, such as by using tape or securement dressing. In other examples, by rearranging the detents or engagement tabs between the left housing frame and the right housing frame, the user can simultaneously apply a compressive force and a proximally directed force on the top lever and a compressive force and a distally directed force on the bottom lever to move the two housing sections relative to one another and the detents on the first frame and second frame to separate.

In some examples, rather than causing two housing sections of the housing to separate to enable separation of the catheter hub from the housing, following successful venipuncture, the housing can be provided with one or more gates at the nose end of the housing. For example, when the catheter hub is advanced against the two gates, detents or engagement that caused them to secure together can separate so that the two gates can swing outwardly to open an exit or a path at the distal end of the housing to then allow separation of the catheter hub from the housing. The two gates can be molded directly to the frames of the housing and each provided with a living hinge to swing open. Alternatively, the gates can instead be molded to a carrier and the carrier is then attached to the housing.

A needle hub having a needle with a needle tip and with an adaptor and a dispenser can be referred to as a piercing and guiding unit, or PG unit. A PG unit may be used with any catheter assembly in which the catheter tube and needle have a relatively longer length, in the order of about 7.5 cm to 20 cm. Such relatively longer catheter and needle are typically found with midline catheters. However, the PG unit of the present invention is not so limited and may in some instances may be used with shorter catheters and needles, typically found in peripheral access catheters, or longer catheters.

Typically following successful venipuncture, the entire PG unit is removed together as a single unit from the catheter tube and catheter hub, which can remain with the patient once placed into a vessel. Where necessary, components of the PG unit can be removed from the catheter hub via piecemeal, starting first with the dispenser, then the adaptor, then the needle hub with the needle, or combinations thereof. Preferably, the guidewire and the needle can be removed together as a unit.

The PG unit, when used with a catheter assembly, with or without an external housing as an extended dwell catheter assembly, would be used with a catheter hub having a catheter tube extending therefrom. The PG unit is used for vascular access then subsequently removed from the catheter following successful venipuncture. For example, a user can first grasp the catheter assembly with the PG unit and guide the tip of the needle through the skin at a suitable insertion site to access a vein or vessel.

Confirmation of proper vasculature access can be confirmed via primary blood flash, i.e., the presence of blood in the needle hub. The user can then retract the needle tip proximally of the catheter tube opening to observe secondary blood flash back, i.e., the presence of blood between the outer diameter of the needle and the inner diameter of the catheter tube. In some instances, the needle can have a through notch formed through the wall of the needle shaft, near the needle tip, or a lengthwise groove by forming a depression lengthwise along the needle shaft. These alternatives allow flashback to be observed between the interior of the catheter tube and the exterior of the needle shaft and can be detected relatively quicker than blood flashback at the needle hub. At the notch, the needle can have a reduced inside diameter or reduced lumen due to the notch formation process. Thus, the notch can also be used as a stop feature to stop distal advancement of the guidewire, such as to grip the change in guidewire diameter section, as further discussed below.

After successful venipuncture is confirmed, the guidewire dispenser is activated by applying digital pressure on the guidewire at the advancing or advancement platform of the dispenser and then sliding or feeding the guidewire distally. The guidewire, such as the tip of the guidewire, may initially be disposed within the hollow needle, may be only initially disposed in the adaptor, or may be only initially deposed in the catheter hub.

Distal guidewire advancement continues until the guidewire has been fully extended within the vessel of the patient. In an example, the amount of extension of the guidewire into the vessel may be controlled by fixing the overall length of the guidewire that can extend at the advancement platform. For example, the guidewire may have two ends, a first end at the distal end and a second end at the proximal end of the guidewire. The length of the guidewire can be selected so that when the second end of the guidewire is moved to the advancement platform, it marks the full extended position of the guidewire. The user can then stop further advancement of the guidewire at that point. Optionally a physical stop, as discussed above and elsewhere, or an indicator may be provided so that when the guidewire is advanced, the stop or the marker indicates how far the first end of the guidewire has extended distally beyond the needle tip. Of course, the user does not have to fully extend the guidewire when using the catheter assembly with the PG unit of the present invention. In some examples, one or more markers or indicators can be used to indicate when guidewire reaches bevel tip at the needle tip, passes through bevel tip, and when the guidewire advances fully out of bevel tip, or to indicate all the foregoing conditions.

The catheter tube can now further be inserted into the vessel and be guided by the guidewire. As the catheter tube, and the catheter hub, moves distally forward and the catheter tube guided by the guidewire into the vessel, the catheter hub separates, or further separates, from the needle hub. The catheter tube and catheter hub can be advanced a sufficient amount over the guidewire to ensure successful placement within the vessel. The PG unit can then be removed or separated from the catheter tube and catheter hub. After removal, the PG unit is separated from the catheter hub and catheter tube.

Where the PG unit is used with or as part of an extended dwell catheter assembly, the method of using the assembly is similar to the process described immediately above except that when the catheter hub is advanced to move the catheter tube over the guidewire, the catheter hub or an attachment to the catheter hub can open the gates at the distal end of the housing to allow separation of the catheter hub from the housing or a user can activate levers, tabs, or buttons to split the housing into two or more housing sub-components to then allows the catheter hub separate from the housing. The PG unit can separate from the catheter hub at the same time, before, or after the housing separates from the catheter hub.

A dispenser in accordance with aspects of the invention has a frame or body, a duct engaged to the body, and a guidewire. The frame has a sideway generally Y-shape configuration with a first branch, a second branch, and a third branch.

The three branches can connect to one another at a focal or connected portion of the frame or body and each branch can have a respective free end that extends away from the focal or connected portion. The free ends are spaced from one another and the frame with the three branches can be unitarily formed.

As shown, the first branch has a free end that generally points in the distal direction, the second branch has a free end that generally points in the proximal direction, and the third branch has a free end that points radially of a lengthwise axis defined by the first and second free ends, and the free end of the third branch optionally also points proximally in addition to pointing radially. In an embodiment, each branch of the frame is elongated and can have surface contours and/or arcuate bends. For example, the first branch can extend distally from the connection portion and can have a reduced thickness section defining a guide platform. The reduced section can be formed by providing a surface that resembles a section of an arc, which has sections of larger width or thickness on either side of the reduced section.

The first branch can have a connection tip at a distal end thereof. The connection tip resembles a reducer and reduces down to act as a slip-on tip or nipple, for connecting to the open proximal end of the adapter. The connection tip of the dispenser can reduce to a first tip section and then further reduced to a smaller diameter second tip section. The size and shape of the connection tip, including the first and second tip sections, are configured to mate with or to a matching female bore of the adaptor.

In an example, the engagement between the two is a taper interference fit, similar to a Luer fit but not necessarily formed to the Luer ISO standard. In yet other examples, the adaptor and the connection tip of the dispenser can be provided with a detent or detent-like engagement. As shown, the connection tip and the receiving bore of the adaptor can include a raised lip or a shoulder and a corresponding recess to form a snap fit. In still other examples, rather than a connection tip with different tip sections, such as different tip diameters, the connection tip can embody a single taper for engaging a corresponding taper of the adaptor, similar to a Luer taper but with non-standard ISO sizes.

The first branch can have a guide passage extending axially through the first branch and through the connection tip. The guide passage is provided for passing the guidewire therethrough and into the adaptor, and into and out of the needle during use for guiding the catheter tube, as previously discussed. For example, the guidewire has two ends, called a first end or distal end and a second end or proximal end.

In a ready to use position, the first end can extend through the guide passage, out the connection tip and into the interior of the adaptor. In an example, the first end or distal end of the guidewire does not enter the needle lumen in the catheter assembly ready to use position to provide flow space for primary blood flashback during needle puncture, as further discussed below.

The dispenser can have a sloped section that inclines in the distal to proximal direction towards the second branch. A guide bracket can be located at the proximal end of the second branch. The guide bracket can have a body that is generally cuboid in shape, such as resembling a rectangular prism, with a slanted or tapered distal end or distal side, which is sloped or tapered. In some examples, the guide bracket has a different cross-sectional shape, such as round or polygonal and the distal side can be more vertically aligned.

A guide passage can be provided axially through the body of the guide bracket to accommodate the guidewire. Elevation-wise, the distal end or side of the guide bracket can seat higher than the entrance to the connection tip at the end of the first branch. This elevation difference can facilitate guidewire dispensing by allowing the dispenser to be gripped while using the thumb to slide the guidewire in the distal direction, as further discussed below. However, the angle of the guidewire extending between the guide bracket at the second branch and the guide tip at the first branch can vary while still providing the dispenser with an ergonomic shape.

The guide bracket at the second branch can be provided with a socket for receiving a first end of the duct. The guide bracket can have a plurality of walls defining a round socket therein. In an example, the duct can embody a round tubing section and the first end of the tubing can engage a round socket of the guide bracket, such as in a friction fit, an interference fit, or a snap fit. Using the duct with the frame is preferred but not required as the guidewire can simply extend out the second branch and dangle when the duct is not used, which is not preferred. As incorporated, the duct receives part of the guidewire therein to prevent kinking or bending of the guidewire. In still alternative examples, a duct having a shorter length than shown can be incorporated and the second end of the duct dangles in the air.

In an example, the second end of the duct and the guidewire dangle from the second branch, with the second end unsupported. Alternatively, the duct has a curved body to curve around so that the second end of the duct projects into the socket of the support bracket of the third branch, in a friction fit, interference fit, or a snap fit. The socket can be round and formed by wall surfaces of the support bracket. The duct in the embodiment shown is therefore supported at both the first end and the second end by the guide bracket and the support bracket, respectively. The support bracket can be practiced without a guidewire passage should the length of the guidewire be sufficiently short and not extend past the opening at the second end of the duct. However, a guidewire passage can be incorporated axially through the support bracket if necessary to accommodate the length of the guidewire.

In still other examples, the socket of the third branch has a pass through so that the second end of the tubing extends through and beyond the socket at the third branch towards central part of the tubing length. The second end can then be held with the central part of the tubing length by one or more clips, as further discussed below. This version of the pass-through socket at the third branch allows the tubing to be looped in a relatively smaller loop than the version with a closed-end socket. This in turn reduces the overall profile of the guidewire dispenser.

The support bracket at the third branch is generally cuboid in shape, such as resembling a rectangular prism, with a slanted or tapered remote end or distal side, which is sloped or tapered. In some examples, the support bracket has a different cross-sectional shape, such as round or polygonal and the distal side can be more vertically aligned.

The body of the dispenser can be provided with a curved or arcuate edge or surface between the third branch and the first branch. Similarly, the body can be provided with a curved or arcuate edge or surface between the third branch and the second branch. The curved edge between the third branch and the second branch of the frame and the curved duct can form a generally round structure having a hollow space or opening for receiving or accommodating the guidewire and for gripping. For example, the curved edge between the third branch and the first branch provides a gripping surface for the user. As shown, the duct and the frame can form a closed loop. In particular example, the duct and the curved edge surface of the frame can define a generally round structure.

During use, the user, in a one-handed use, can grip the dispenser by placing the middle finger, ring finger, and pinky into the hollow interior of the dispenser and up against the curved edge. The index finger can grip around the curved edge between the third branch and the first branch, opposing the inner curved edge. The thumb can then be placed over the upper side of the dispenser, over the sloped section, and above the guidewire and the guide platform.

To dispense or activate the guidewire, the user uses the thumb to press the guidewire against the surface at the guide platform with both a downward force against the surface and a distally directed force to move the guidewire in the distal direction. This will move the first end of the guidewire through the needle and out the distal end thereof to help guide the catheter tube.

Total guidewire movement out the needle tip can be controlled by controlling or sizing the total length of the guidewire so that when the second end of the guidewire is pushed into connection tip at the first branch, no further guidewire is available to dispense. In other examples, the second end of the guidewire, or some part of the guidewire, can be provided with a catch, such as a hook, an enlarged section, such as a larger diameter, a knot, etc., that catches on the frame of the dispenser or against the reduced lumen opening at the crimp of the needle after the guidewire has dispensed some finite amount to prevent the guidewire from further distal movement out the connection tip and/or out the needle tip. Obviously, while the curved edges of the frame are preferred for the reasons noted, they can be modified to be less curved or even have straight sections and still provide the general functions described.

In an example, the tip of the adaptor projects into the interior space of the needle hub and the distal opening of the tip located over the proximal end of the needle, which has a blunt end. In an example, the bore or cannula channel at the distal opening of the tip is sized to have a size-on-size fit with the needle proximal end with a loose fit, such as having a small clearance between the two, being optionally employable. A flared inlet or entrance can be provided at the distal opening of the tip. The flared inlet allows the tip to readily slide over the needle to receive the proximal end into the bore of the tip and helps to compensate potential slight misalignment.

The tip can be provided with a modified Luer taper to engage the needle hub in a Luer fit at the proximal opening of the needle hub. The tip can be elongated so that the distal end of the tip can reach and project over the needle proximal end to receive the needle inside the bore of the tip.

An adaptor provided in accordance with aspects of the invention has a body comprising a base and a tip with a shoulder located between the two. Exteriorly, the base can be provided with a plurality of slats or sidewalls that are inter-connected and angled relative one another. The walls or slats can connect along respective edges. At the end of at least one of the slats, a flange or tab is provided. The flange or tab can be continuously formed with the slat from which it extends.

The flange or tab can have at least three unobstructed three side edges. In other examples, the flange or tab can be rounded, such as being partially circular, or can have more than three edges. There can be more than one flange or tab incorporated at the base. In a preferred embodiment, at least two spaced apart flanges or tabs are incorporated at the base. When two spaced apart flanges are incorporated, the tip of the dispenser placed therebetween

The tip of the adaptor can be generally frustoconical in shape with one or more reduced tip sections. For example, the tip can have a first tip section with a first size, a second tip section with a second size, a third tip section with a third size, and a fourth tip section with a fourth size. However, the tip can have other bore shapes and still encompasses aspects of the invention. In other examples, there can be fewer or more than the number of sections depending on the fit desired with the interior of the needle hub and the reach of the tip inside the needle hub to either surround or be adjacent the needle proximal end. In other examples, the tip is provided with a single tapered profile.

The two flanges or tabs extending at the base of the adaptor can be opposed along a lengthwise axis and each can comprise an interior surface having a lip or a surface engagement feature. The gap distance between the two tabs can be relatively smaller than the inside diameter of the base. This relatively smaller dimension between the two flanges or tabs can be selected to engage a lip or corresponding surface on the connection tip of a guidewire dispenser.

Internally, the adaptor can have a bore that is open so that the proximal end is in fluid communication with the distal end of the adaptor. In an example, the bore has different bore sections with different bore diameters. The different bore diameter sections can be selected or sized to fit with selected connection tip of a guidewire dispenser. The bore can have a first bore section having a first diameter for coupling to the first tip section of the dispenser and a second bore section having a second diameter for coupling to the second tip section of the dispenser. Distal of the second bore section, a third bore section can be provided.

The third bore section, when incorporated, can have a frustoconical shape that necks down to a guidewire passage and then expands in diameter to the cannula channel, which can have a larger cross-sectional space than the guidewire passage as the needle has a larger outside diameter than the guidewire diameter. At the distal end of the adaptor, the flare inlet having a tapered end most edge is provided to facilitate placement of the cannula channel over the needle blunt proximal end, as previously discussed.

The cannula channel can have a non-circular space defined by three generally straight sides connected to one another by arcuate connecting sections. If the proximal end of a needle is placed into the cannula passage, the needle proximal end would resemble a disc-shaped and would contact the three generally straight sides of the cannula channel. The needle bore would align with the guidewire passage shown, in the center of the disc-shaped structure. The contact between the exterior of the needle and the three generally straight sides can generate a slight interference fit to ensure a firm connection between the cannula channel and the needle. Each arcuate connection section and the needle exterior define a bypass gap at the cannula channel. The tip of the adaptor and the needle at the cannula channel define three bypass gaps. In other examples, there can be more than three straight sides and corresponding number of arcuate connection sections.

In use during initial needle puncture, a guidewire occupies the guidewire passage at the tip of the adaptor, but the guidewire first end does not project into the needle lumen, at the needle proximal end. Thus, when primary blood flashback flows proximally out of the needle proximal end, the blood first flows into the cannula channel but cannot flow proximally into the bore at the base or does not materially or substantially flow into the bore at the base due to the restriction. This is because the guidewire occupies the guidewire passage and the clearance between the guidewire and the bore diameter at the guidewire passage is too restrictive for blood to flow, or to freely flow, thereacross.

However, because the bypass gaps can be sized for blood flow, such as by providing a sufficient flow passage or clearance at each bypass for a given fluid viscosity, blood entering the cannula channel can flow out through the bypass gaps and into the interior space of the needle hub. A practitioner can thus detect primary blood flashback at the interior space of the needle hub after blood flashback flows into the cannula channel of the adaptor and then reverses flow and flow out through the bypass gaps and into the interior space of the needle hub to be detected visually through the wall surfaces of the needle hub, such as by perceiving the blood red color within the interior space. In some examples, the adaptor can be made with a translucent material that is at least partially transparent and blood flashback can be observed through the walls of the adaptor.

In an alternative embodiment, an adaptor has a body comprising a base and a tip. The base can similarly have a plurality of slats or sidewalls joined along respective edges. One or more flanges or tabs can extend proximally of the base with each flange or optionally incorporating engaging features for engaging the guidewire dispenser, as previously discussed. In an alternative embodiment, the exterior at the base can be one continuous generally round cylinder instead of distinct slats.

The tip of the present adaptor can be provided with different exterior tip sections as well as different interior tip sections. In an example, the distal end most of the tip has a bore that is provided with a shaped internal bore, resembling a frustoconical shaped section. However, the tip can have other bore shapes and still encompasses aspects of the invention. The shaped internal bore is distal of a first bore section and second bore section and has a reduced tip defining a guidewire passage. The guidewire passage is sized and shaped to accommodate a guidewire passing therethrough but is otherwise too small to accommodate a needle proximal end.

Thus, in an exemplary embodiment, the adaptor does not incorporate a cannula channel distal of a guidewire passage for receiving a needle proximal end. In use, the distal most tip of the adaptor can be spaced from the proximal end of the needle and any primary blood flashback flowing through the needle lumen can simply flow directly into the interior space of the needle hub. In some examples, the adaptor can have a valve and a tip with a cannula channel that receives the proximal end of the needle.

In an alternative embodiment, the present adaptor can be lengthened and incorporate a structure distal of the guidewire passage to define a cannula channel. If incorporated, the adaptor can receive the proximal end of a needle in the cannula channel when the adaptor is engaged to the needle hub. Blood flashback into the cannula channel of the modified adaptor with a cannula channel can therefore reverse and flow out of bypass gaps at the modified tip.

The base of the present adaptor can be provided with a valve chamber having a valve located therein. In an example, the base is provided with internal shoulders to form varying internal chamber sizes or sections, including a first bore section or chamber, a valve chamber, and a reduced chamber located between the first bore section and the valve chamber.

In an example, the first bore chamber is configured to receive a connection tip of a guidewire dispenser and the reduced chamber is configured to accommodate a reduced tip section of the connection tip, as further discussed below. In some examples, the base can be a continuous or can have a generally cylindrical structure without distinct slats and edges formed between the slats.

A valve can be placed in the valve chamber and held therein by a distal shoulder and a proximal shoulder. In an example, the valve can have a valve disc and a valve skirt extending from the valve disc. The distal surface of the valve disc can press or contact the distal shoulder and the proximal end of the valve skirt can press or contact the proximal shoulder to position the valve therebetween. In some examples, the valve can be provided with a valve disc without a skirt section depending therefrom and the internal bore of the adaptor modified accordingly to hold the valve without the skirt section.

In an example, a valve usable with the adaptor can have a valve disc and an integrated skirt depending therefrom. In the example shown, the valve disc and the skirt section are unitarily formed. The skirt section can be viewed as a generally cylindrical length having an open proximal end, having a proximal most edge.

The valve disc can have a plurality of slits or valve disc slit portions defining a plurality of valve flaps. For example, the valve can have three slits defining three flaps. However, the valve can be practiced with one slit defining two flap sections or more than three slits and three flaps. The three slits can meet at a focal or central point. The slits are preferably equal in length but not required to be equal and the slits stop short of the outer diameter of the valve disc. In the present embodiment, the wall thickness of the valve disc, in the axial direction, is generally constant, without varying surface features. However, distinct surface features can be incorporated.

In some examples, the valve skirt can have a generally constant outside diameter (OD). The valve skirt can have a generally constant thickness so that the inside diameter of the skirt can be generally constant. In alternative embodiments, the thickness can vary so that the outside diameter of the skirt section can be constant while the inside diameter can vary.

In another embodiment, the valve has a valve disc and a valve skirt depending therefrom. The valve disc can have a plurality of slits or valve disc slit defining a plurality of valve flaps. In the present embodiment, the wall thickness of the valve disc, in the axial direction, has a distinct first portion and second portion. The first portion can have a first thickness and the second portion can have a second thickness and wherein the first thickness can be greater than the second thickness, measured orthogonal to a medial plane passing through the diameter of the valve. The second portion with the second thickness can have a substantially constant thickness but can optionally include varying thicknesses along a cross-section of the valve, at the second portion.

In an example, the second portion can be formed by recessing the distally facing surface of the valve disc, the proximally facing surface of the valve opposing the distally facing surface, or both the distally and proximally facing surfaces of the valve disc while the first portion retains substantially the full width or thickness of the valve between the proximally and distally facing surfaces. In an example, the recesses at the second portion can embody undercuts formed into the valve.

The surface appearance between the first and second portions can resemble a three-leaf clover. The three-leaf clover can be present on the distally facing surface, the proximally facing surface, or both surfaces of the valve. In other examples, the surface appearance of the proximally and/or distally facing surfaces can have varying contours so that the three-leaf clover can have contours of varying curves, lines, and edges. The surface features can facilitate flexing and sealing of the valve at the slits.

A PG unit having a dispenser, an adaptor, and a needle hub having a needle extending therefrom can be practiced with an adaptor having a valve. The valve can have a valve disc and a valve skirt. The distal most tip of the dispenser with a valve located therein can be spaced from a proximal end of a needle. However, the tip of the adaptor can include a cannula channel and the needle proximal end can be located in the cannula channel.

An alternative guidewire dispenser can have a frame or body modified so that the guidewire extends generally horizontally and generally coaxial with the needle through the guide platform of the frame. The frame can be provided with a guide passage at the sloped section that inclines towards the guide bracket at the second branch. This arrangement lowers the angle of insertion of the guidewire as the guidewire extends towards the connection tip of the dispenser and into the adaptor.

The connection tip of the dispenser can project into the receiving space or bore at the base of the adaptor and the one or more tabs engage corresponding detent surfaces on the dispenser to maintain a firm engagement between the adaptor and the dispenser. Optionally the engagement can be an interference fit without use of the flanges or tabs.

As shown, the distal tip section of the dispenser can be located in the interior space defined by the skirt of the valve but does not push through the valve disc to deflect the valve flaps. In an example, the distal tip section of the dispenser is spaced from the proximally facing surface of the valve disc. The guidewire extends through the slits of the valve disc such that the first end of the guidewire is located in the bore of the adaptor and recessed from the distal most tip of the adaptor. The location of the first end of the guidewire can be practiced in the ready to use position of the catheter assembly.

The tip of an adaptor can be positioned in the interior space of the needle hub and the connecting tip of the dispenser is located inside the bore at the base of the adaptor. The distal most tip of the adaptor can be spaced from the needle proximal end such that a gap is provided therebetween. The first end of the guidewire is located in the bore of the adaptor and recessed from the distal most tip of the adaptor. The adaptor can have a valve located therein.

Following initial vascular access, blood flows in the proximal direction through the lumen of the needle and out the proximal opening of the needle. Because of the gap between the needle and the tip of the adaptor, some of the blood spills into the interior space of the needle hub, which can then be detected visually through the walls of the needle hub as primary blood flashback. Some blood may also flow through the distal opening at the tip of the adaptor, which can be a guidewire passage. If so, blood flow through the bore of the adaptor can be restricted or stopped by the valve located inside the adaptor. This way, a practitioner can take appropriate time to place the guidewire into the vein and then slide the catheter tube over the guidewire to place the catheter tube deep into the vein without feeling rushed due to the outpouring of blood out through openings at the adaptor and/or dispenser for an adaptor without the seal. Following successful venipuncture, the PG unit, which includes a needle hub, an adaptor, and a guidewire dispenser, can be removed from the catheter hub and catheter tube. The components can be removed as a single integrated unit or can be removed individually.

A protective cap can be provided at the tip of the adaptor. The assembly having the protective cap, adaptor, and guidewire dispenser, which has a guidewire and a duct or tubing, can be provided in a shipping package, such as blister pack or a thermoplastic pouch, and be commercially available as a unit.

The protective cap can have a first end and a second end and formed as a thermoplastic protective sleeve. The first end can have a bore that is sized and shaped to snap fit over the tip of the adaptor. Optional detent engagement and/or friction fit between the two can be practiced. The second end of the protective cap be a closed end or can have an opening. The protective cap can be removed from the adaptor and disposed prior to using the adaptor and guidewire dispenser unit.

An alternative guidewire dispenser can have a frame or body that is modified so that third branch can have a C-channel rather than a socket. The C-channel of the modified third branch can be a pass-through clip having a slot. That is, the duct does not end at the third branch but is routed through the clip or C-channel at the third branch so that the second end of the duct is away or remote from the C-channel.

The duct can snap fit into the clip by placing the duct over the slot and then pressing the duct through the slot to be seated within the C-channel. Less optimally, the second end of the duct can be routed through the C-channel by inserting into the end opening and then pushing the second end through.

An intermediate retaining clip can be provided to hold parts of the mid-section of the duct together. In the example shown, the intermediate retaining clip has an integrated or a unitarily formed body with two slots for receiving two different sections of the duct. The intermediate retaining clip can be viewed as two C-channels that are formed side-by-side, with a common center side. Both C-channels can be pass through in that the tubing does not terminate at any of the C-channels.

A second intermediate clip can be used to support additional sections of the tubing or duct. For example, the second intermediate clip can be placed adjacent the first intermediate clip, either in contact with or spaced from the first intermediate clip, to support the duct near the second end, with the second end passing through the second intermediate clip.

Alternatively or additionally, an end clip can be provided to support the duct or tubing, and particularly the second end of the duct. The end clip can be viewed as a C-channel and a modified C-channel that are formed side-by-side, with a common center side. The end clip has a C-channel that has a pass-through slot and a closed slot in which an end wall is provided. The end wall can prevent the second end from passing through the modified C-channel.

By using one or more intermediate clips and optionally with an end-clip in combination with the pass-through C-channel at the third branch, the tubing can be coiled to have an overlapping section or sections. That is, the tubing can be coiled with over one revolution of loop. This allows the duct and the frame to form a closed loop that is smaller in profile than a similar sized dispenser and duct without C-channels. This in turn can reduce the packaging size and storage space.

Methods of making and of using the catheter assemblies and components thereof, such as with one or more of an outer housing, with a guidewire, with a guidewire dispenser, and with an adaptor, are within the scope of the present invention.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a perspective view of a catheter assembly located inside a housing, the catheter assembly comprising a catheter with a catheter tube and a needle hub with a needle.

FIG. 2 is a side partial cross-sectional view of a catheter assembly usable with the housing of FIG. 1.

FIG. 3 is a piercing and guiding unit, or PG unit, comprising a needle, a needle hub, an adaptor, and a guidewire dispenser having a guidewire.

FIG. 3A is a blown-up view of the dispenser of FIG. 3.

FIG. 4A is a partial cross-sectional side view of a PG unit engaged to a catheter hub.

FIG. 4B is an enlarged partial cross-sectional side view showing the tip of the adaptor of FIG. 4A located inside the interior space of a needle hub.

FIG. 5 is a perspective view of an adaptor.

FIG. 6A is a cross-sectional side view of the adaptor of FIG. 5; FIG. 6B is a cross-sectional end view taken along line D-D of FIG. 6A; and FIG. 6C is an enlarged view of section E of FIG. 6B.

FIGS. 7A and 7B are side elevation view and cross-sectional side view thereof.

FIG. 8 is a side elevation view of PG unit.

FIG. 9 is a cross-sectional side view of an adaptor with a valve located therein.

FIG. 10 is a valve usable with the adaptor of FIG. 9.

FIG. 11 is a valve usable with the adaptor of FIG. 9.

FIG. 12A is a PG unit having an adaptor with a valve located therein and FIG. 12B is an enlarged view of the guidewire dispenser and adaptor of FIG. 12A.

FIG. 13 is an enlarged view of a PG unit with an adaptor having a valve and direction of blood flow.

FIG. 14 is a side view of an adaptor and guidewire dispenser with a protective cap located over the tip of the adaptor.

FIG. 15 is a partial perspective view of a piercing and guiding unit or PG unit.

FIG. 16 is a rear perspective view of the adaptor of FIG. 15, separated from the other components.

FIG. 17 shows a front perspective view of the adaptor of FIG. 16.

FIG. 18 shows an end view of the adaptor of FIG. 17.

FIG. 19 is an enlarged end view of the cannula channel of the adaptor of FIG. 18.

FIG. 20 is a front perspective view of an adaptor with a plurality of gas channels.

FIG. 21 is a front-end view of the adaptor of FIG. 20.

### DETAILED DESCRIPTION

The detailed description set forth below in connection with the appended drawings is intended as a description of the presently preferred embodiments of catheter assemblies and components thereof provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features. Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Generic identifiers, such as first and second, are understood to merely identify different components by their generic names only to keep track of the presence but are not structurally limiting. Further, while one component may be referred to as a first and another as a second, the particular reference to the different components can be reversed or changed and not structurally limiting. The term can or may is meant to convey features that can or may be included but not required.

Broadly speaking, extended dwell catheter assemblies described herein each comprises a housing and a catheter assembly. The housing is configured to accommodate the catheter assembly during initial puncture, through catheter advancement, through separation of the needle hub from the catheter hub, and then activating to enable separation of the catheter hub from the housing and allowing the catheter to remain with the patient.

The housing and the catheter hub can separate through a variety of different mechanisms. For example, the housing can split into two or more housing parts to release or separate from the catheter hub, the housing can have parts that swing or pivot to open a path for the catheter hub to separate, the housing can have end cap components that swing open to open the gate opening to allow the catheter hub to exit the housing, or the housing can have a gate assembly that attaches to the distal end of the housing that can be activate to open once the catheter hub is advanced distally within the housing to slide the catheter tube deep into the vein, among other options.

If a gate assembly is used with the housing, the gate assembly can have two gates with each gate located on each of the two gate housing bodies or housing sections, which housing sections can join, and wherein each gate can swing open via a living hinge. The two gates can engage via male detents and receiving slots. The two gates can engage via a hook arm and a female detent. The unique housing designs for use with catheter hubs allow for use with standard or existing catheter assemblies without the need for, although optional, specifically designed catheters. Thus, standard catheters with extended lengths and midline catheters with approximately 8-10 cm catheter lengths are readily usable with the housings of the present disclosure, with or without a guidewire. Exemplary extended dwell catheter assemblies and housings noted hereinabove are disclosed in provisional application Ser. No. 62/893,382, filed Aug. 29, 2019, entitled “Extended Dwell and Midline Catheters and Related Methods”, the contents of which are expressly incorporated herein by reference.

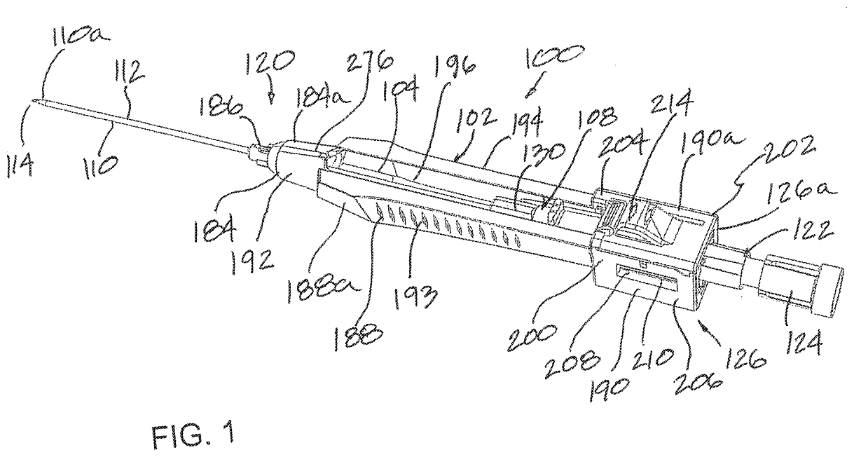

With reference now to FIG. 1, a perspective view of an exemplary embodiment of an extended dwell catheter assembly 100 in accordance with aspects of the invention. In the example shown, the extended dwell catheter assembly 100 comprises a housing 102 having a receiving space or interior space 104 having a catheter assembly 108 located therein. As shown, the catheter assembly 108 has a vent plug 124 engaging the proximal opening of the needle hub 122. In alternative embodiments, as further discussed below, the vent plug 124 is replaced with an adaptor and a guidewire dispenser, which is coupled to or engaged to the adaptor. The catheter assembly 108 with the adaptor and guidewire dispenser may optionally be used with the housing 102, or without the housing.

The catheter assembly 108 has a catheter tube 110 and a needle 112 located inside the lumen of the catheter tube extending out a distal end 120 of the housing 102. A needle tip 114 of the needle extends out a distal opening 110a of the catheter tube 110. The catheter tube 110 is attached to a catheter hub 130 and the needle 112 is attached to a needle hub 122. The needle hub 122 is shown extending out a proximal end 126 of the housing 102. The needle hub 122 can include a vent plug 124, which can engage the proximal opening of the needle hub 122, such as in a Luer fit.

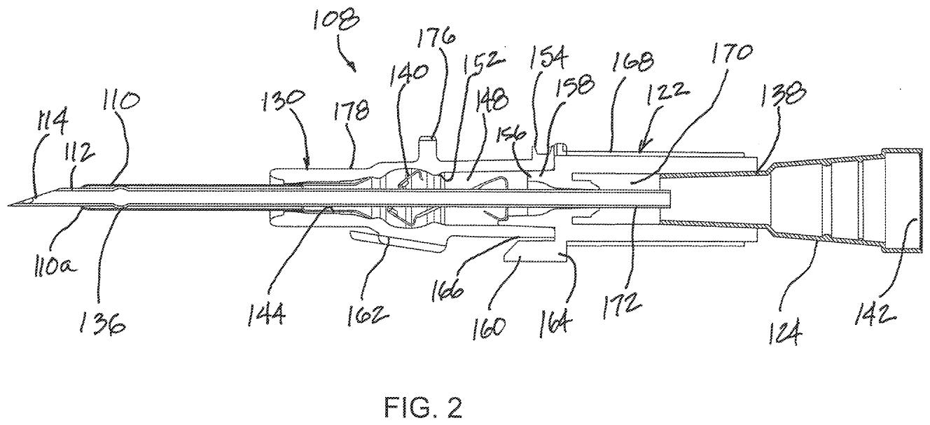

With reference now to FIG. 2 in addition to FIG. 1, a cross-sectional side view of the catheter assembly 108 of FIG. 1 is shown outside or away from the housing 102. The presently shown catheter assembly 108 and similar catheter assemblies may be used with the housing 102 to form an extended dwell catheter assembly in accordance with aspects of the present invention. Optionally, a guide wire may be incorporated to assist in inserting, positioning, and moving a catheter into the vasculature of a patient, as noted above and further discussed below.

In an example, the catheter assembly 108 comprises a first hub or catheter hub 130 having an over-the-needle tube or catheter tube 110 attached thereto and a second hub or needle hub 122 having a needle 112 attached thereto. The catheter tube 110 has a lumen or bore for receiving the needle 112 and has a distal opening or distal end opening 110a and the needle tip 114 of the needle 112 extends distally of the distal opening 110a in a ready to use position, in which the catheter assembly is ready for venipuncture or for accessing the vasculature of a patient. The catheter assembly 108 can also be called a needle assembly.

The needle 112 can include a change in profile 136 located proximally of the needle tip 114 for use with a needle guard 140. The needle tip 114 extends distally of the distal opening 110a, which can be called a distal end opening, of the tube or tubing 110 in the ready to use position with the change in profile 136, if incorporated for use with a needle guard, located proximally of the distal end opening 110a. The change in profile 136 can be a crimp, a bump, or a material build-up having a different profile than other diameter sections of the needle shaft. As used herein, the term proximal is understood to mean an end or side closer to the practitioner and the term distal is the opposite end or side.

The change in profile 136 can be used to interact with a needle guard 140 during retraction of the needle 112 following successful venipuncture, as further discussed below. A vent plug 124 is disposed at the proximal open end or proximal opening 138 of the second hub or needle hub 122. The vent plug 124 can have a vent filter 142 at a proximal end thereof, which is conventional. As shown, the catheter tube 110 is attached to the first hub or catheter hub 130 by a ferrule or bushing 144.

The needle guard 140 can be provided in the interior cavity 148 of the first hub or catheter hub 130 for covering the needle tip 114 in the protective position. When incorporated, the needle guard 140 can be one of the needle guards disclosed in U.S. Pat. No. 6,616,630, the contents of which are expressly incorporated herein by reference. In some examples, the needle guard 140 can be omitted. If so, the change in profile 136 on the needle can optionally be omitted. In some examples, the change in profile 136 can interact with the guidewire to limit distal travel of the guidewire, as further discussed below. In still other examples, a needle guard can be included without also including a change in profile on the needle. In yet other examples, the needle guard 140 can be located in a third housing between the first hub 130 and the second hub 122. An exemplary needle guard located in a third housing is disclosed in U.S. Pat. No. 8,597,249, the contents of which are expressly incorporated herein by reference. In still other examples, the needle guard can include multiple arms or fingers extending in both a first direction and an opposite second direction as disclosed in U.S. Pat. No. 9,387,307, the contents of which are expressly incorporated herein by reference. For example, the needle guard can include a first protector body surrounded by a second protector body.

In an example, the needle guard can be made from a metal material and can have a resilient portion that can generate a biasing force. The needle guard can comprise a wall surface that is positioned to a side of the needle and is movable distal of the needle tip to guard the needle tip from accidental contact therewith. The wall surface can be a needle trap and can move directly in front or distal of the point of the needle tip. The wall surface can alternatively be a second protector body that surrounds the first protector body.

As shown, the needle guard 140 can include a proximal wall and two arms extending distally of the proximal wall. The two arms can intersect one another in the ready to use position of FIGS. 1 and 2, and when viewed from the side view of FIG. 2, and in the protective position in which the needle guard covers the needle tip, again as viewed from a side. In some examples, the two arms of the needle guard 140 can extend in a distal direction without intersecting one another. Two distal walls, one on each arm, can be incorporated to block the needle tip. The two distal walls can be biased outwardly by the needle 112 in the ready to use position of FIG. 2 and disposed distally of an internal change in profile 152 inside the first hub 130 in the ready to use position. The internal change in profile 152 can be a reduced diameter section located next to an inside diameter section of a larger dimension. The elbows on the two arms of the needle guard can have a dimension that is larger than the inside diameter at the internal change in profile 152 to restrict proximal movement of the needle guard from the catheter hub until the two arms are no longer biased outwardly by the needle 112 or until the dimension at the two elbows are reduced.

Each distal wall of each arm can include a curved lip to facilitate relative movement between the needle guard 140 and the needle 112. When in the ready to use position, the dimension measured between the two joints or elbows, at the intersection between each arm and its respective distal wall, is larger than the inside diameter of the internal change in profile 152, which prevents the needle guard 140 from moving proximally thereof due to the relative dimensions. Once the needle tip 114 moves proximally of the two distal walls and therefore no longer biased outwardly by the needle, the two arms are allowed to move radially inward and one or both distal walls close over the needle tip to block the needle tip in a protective position. At this point, the dimension between the two joints or elbows decreases and is smaller than the internal dimension of the internal change in profile 152, which then allows the needle guard 140 to move proximally and be removed from the catheter hub with the needle. In alternative embodiments, the dimension between the two joints can be equal to or slightly larger than the internal dimension of the internal change in profile 152 and the needle can still move proximally by flexing or squeezing the two joints to get through the internal change in profile 152.

The first hub 130 has a proximal opening 156 having a nose section 158 of the second hub 122 disposed therein. The proximal opening 156 of the first hub has a female Luer for receiving a male Luer tip, such as a syringe, an IV tubing connector, a Luer extension set, etc. External threads 154 can be provided on the exterior surface at the proximal end for threaded engagement with a threaded collar of a male threaded Luer tip. A pair of stabilizing wings 162 can extend radially of the first hub 130 to facilitate securement or anchoring of the first hub 130 to a patient following successful venipuncture. Optionally the wings can be omitted. The first hub 130 can embody a standard IV catheter hub without an injection port. In other examples, the first hub can include an injection port or an integrated extension set. If the wings are not present on the first hub or catheter hub 130, then the bottom of the first hub can have a flattened surface angled at between 2 to 15 degrees from the through axis of the first hub.

The first hub 130 is removably secured to the second hub 122 by receiving the nose section 158 of the second hub in the proximal opening 156 thereof. A flange or extension 160 can be provided on the second hub 122 and together with the nose section 158 define a gap 166 having part of the first hub located therein. Optionally the flange or extension 160 can be omitted and a stub 164 or other surface mating features provided. The stub 164 can be the male part of a key fitting in a gap through or partially into the Luer threads to prohibit rotation of the second hub 122 relative to the first hub 130. The proximal facing end surface of the first hub abuts the distally facing surface of the step in the outside surface of the second hub which is at the proximal end of the nose section 158, which is inside the first hub. This abutment can set the amount of protrusion of the needle tip 114 out of the catheter tubing 110a. The stub 164 can be an optional structural feature.

The second hub 122 has a body 168 having an optional interior cavity 170 having the proximal end 172 of the needle 112 projecting there-into. The interior cavity 170 can function as a primary blood flashback chamber. The proximal opening 138 of the body 168 can have a female Luer for receiving the vent plug 124, a syringe tip, or an adaptor therein and the exterior can have threads or be without threads. In an example, the exterior of the body 168 of the second hub 122 is generally cylindrical and without threads. The first and second hubs, or catheter hub and needle hub, respectively, can be made from plastic materials, such as by plastic injection.

A push tab 176 can be provided with the first hub or catheter hub 130. The push tab 176 can be located on an exterior surface 178 of the first hub 130 at a location distal of the exterior threads 154 so as to avoid interfering with the exterior threads. For example, the push tab 176 should be positioned sufficiently distally of the exterior threads 154 so that when a threaded male connector, such as a syringe type with a threaded collar, is connected to the threads of the first hub, the push tab 176 does not interfere with the connection. In some examples, the push tab 176 should be positioned sufficiently distally of the exterior threads 154 so that when the push tab 176 is folded over, the push tab does not interfere with the connection.

In some examples, the catheter assembly 108 can incorporate a valve and a valve opener. For example, a valve and a valve opener can both be located within the catheter hub 130. In use, the valve opener can be pushed distally into the valve to open the one or more slits formed with the valve and deflects the flaps of the valve to open a fluid passage through the valve for fluid flow. An exemplary catheter assembly with a valve and a valve opener is disclosed in U.S. Pat. No. 8,333,735, the contents of which are expressly incorporated herein by reference. The valve opener can have a nose section having a bore and a plunger section located proximally of the nose section. The nose section can be pushed into the valve to open the one or more slits of the valve, such as three slits defining three flaps, to open the valve for fluid flow. The plunger section of the valve opener can comprise two or more plunger components having a gap therebetween. The gap can allow fluid to flow through the plunger components and through the bore in the nose section.

With reference again to FIG. 1 in addition to FIG. 2, the housing 102 of the present extended dwell catheter assembly 100 comprises a left frame or first frame 193 and a second frame or right frame 194 attached to one another. Each frame comprises a nose portion 184a, a body portion 188a, and a base portion 190a. The two nose portions 184a can join to form a nose end 184, the two body portions 188a can join to form a body 188, and the two base portions 190a can join to form a base end 190. In an example, the nose end or nose portion 184 is provided with a nose tip 186 having an elongated cylindrical like structure having a bore for accommodating the catheter tube 110 and needle 112, which is located within the catheter tube. The nose end or nose portion 184 can further include a nose base 192, which has an enlarged portion formed with the tip 186, and a tapered distal portion. A plurality of support ribs or fins can be provided at the intersection between the tip 186 and the nose base 192. In other examples, the nose end 184 can be a spherical or rounded dome having an opening to accommodate the catheter tube and needle.

The body 188 has an interior space 104 for accommodating a catheter assembly, which can be the catheter assembly 108 described with reference to FIG. 2. A body opening or first opening 196 opens into the interior space 104. As shown, the body opening 196 extends from the nose end 184 to the base end 190.

In an example, a second opening opposing the first opening 196 is provided with the body 188 so that the interior space 104 can be accessed from the two body openings. Thus, the body therefore can have a through passage defined by the two body openings 196. In other examples, the body 188 only has a single opening 196 leading to the interior space 104 as shown with the opposing side being solid or is enclosed by wall surfaces of the first frame and the second frame. Optionally the enclosed side can have small windows or openings for access or venting but not a full opening, like that of body opening 196.

The base portion or end 190 extends from the body 188 and has a plurality of sides. In an example, there can be four sides 200, 202, 204, 206 defining an interior space 208, which is open to the interior space 104 of the body 188. In other examples, the base portion or end 190 can embody a generally elongated cylinder or a non-circular cylinder. For discussion purposes, side 204 and side 206 can be identified as the top side and the bottom side, respectively. Side 200 and side 202 can be identified as the left side and the right side, respectively. The sides may also be understood as the first side 200, the second side 202, the third side 204, and the fourth side 206. These specific nomenclatures are given so as to enable calling out a particular side relative to other sides only but are not limiting unless the context indicates otherwise.

The base portion or end 190 can have an open proximal end to allow the needle hub 122 to extend proximally thereof, proximal of an end surface 126a, in the ready to use position of FIG. 1. As shown, the first side 200 and the second side 202 of the base end 190 can each have an opening 210 to allow viewing and accessing the interior space 208 of the base end 190, such as to facilitate or assist assembling of the catheter assembly 108 therein, as further discussed below.

The top side 204 of the base portion or end 190 can be provided with a top lever 214 and the bottom side 206 can be provided with a bottom lever (not shown). When a user simultaneously applies a compressive force and a distally directed force on the top lever 214 and a compressive force and a proximally directed force on the bottom lever, the user can cause the left housing frame 193 and the right housing frame 194 to move relative to one another and the detents on the first frame 193 and second frame 194 to separate so that the housing can then separate into separate housing components, i.e., the left frame 193 and the right frame 194. In other words, by manipulating the top lever 214 and the bottom lever, the user can cause the housing 102 to separate into two or more housing components along the separation line 276. The separation of the housing 102 into housing components can then expose the catheter assembly 108 to separate from the housing 102 following successful venipuncture so that the catheter hub 130 (FIG. 2) can then be secured to an IV line and secured to the patient, such as by using tape or a securement dressing. In other examples, by rearranging the detents or engagement tabs between the left housing frame 193 and the right housing frame 194, the user can simultaneously apply a compressive force and a proximally directed force on the top lever 214 and a compressive force and a distally directed force on the bottom lever 216 to move the two housing sections relative to one another and the detents on the first frame 193 and second frame 194 to separate.

In some examples, rather than causing two housing sections of the housing 102 to separate to enable separation of the catheter hub 130 from the housing 102, following successful venipuncture, the housing can be provided with one or more gates at the nose end 184 of the housing. For example, when the catheter hub 130 is advanced against the two gates, detents or engagement that caused the two gates to secure together can separate so that the two gates can swing outwardly to open an exit or a path at the distal end of the housing to then allow separation of the catheter hub from the housing. The two gates can be molded directly to the frames of the housing and each provided with a hinge to swing open. Alternatively, the gates can instead be molded to a carrier and the carrier is then attached to the housing.

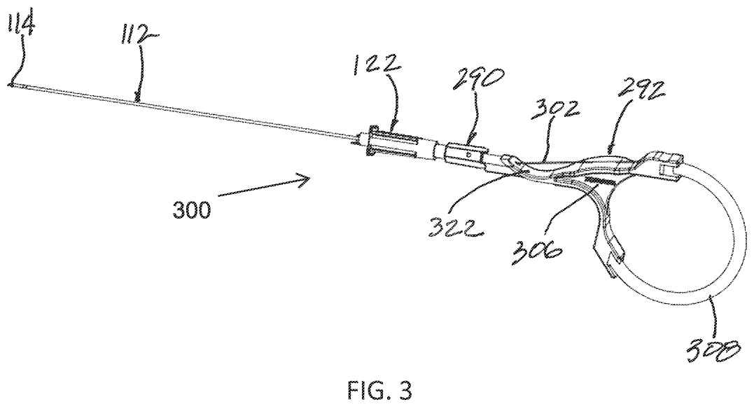

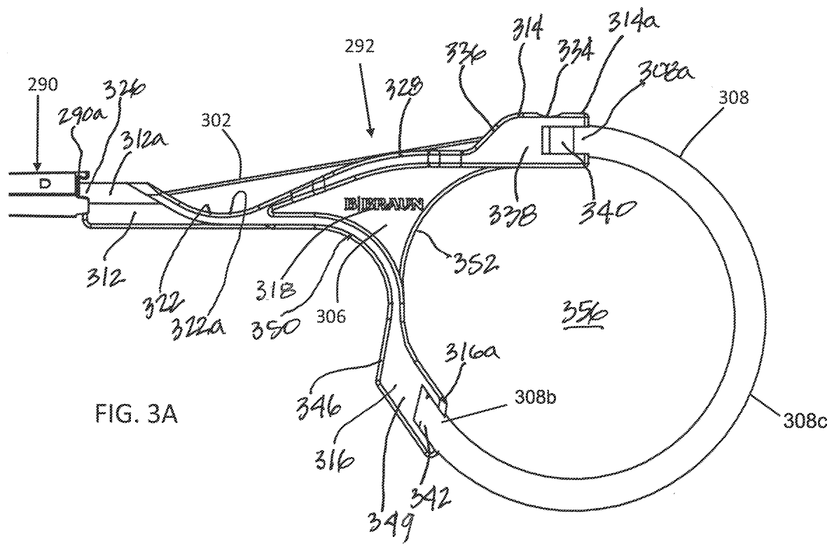

With reference now to FIG. 3, a needle hub 122 having a needle 112 with a needle tip 114 is shown with an adaptor 290 and a dispenser 292. The adaptor 290 may also be spelled or referred to as an adapter. The combination shown in FIG. 3, which has the needle, needle hub, adaptor, and dispenser, may be referred to as a piercing and guiding unit 300, or PG unit 300. A PG unit 300 may be used with any catheter assembly, such as any catheter, in which the catheter tube and needle have a relatively longer length, in the order of about 6.0 cm to 20 cm. Such relatively longer catheter and needle are typically found with midline catheters. However, the PG unit 300 of the present invention is not so limited and may in some instances may be used with shorter catheters and needles, typically found in peripheral access catheters. Typically following successful venipuncture, the entire PG unit 300 is removed together as a single unit and the catheter hub and catheter tube remain with the patient. Where necessary, components of the PG unit 300 can be removed from the catheter hub via piecemeal, starting first with the dispenser 292, then the adaptor 290, then the needle hub 122 with the needle 112, or combinations thereof. Preferably, the PG unit 300 is removed together as a unit instead of piecemeal.

The PG unit 300 of FIG. 3, when used with a catheter assembly, with or without an external housing 102 (FIG. 1), would replace the needle 112, needle hub 122 and vent plug 124 shown in FIG. 2. As shown, the PG unit 300 does not utilize a vent plug, which has been removed from the needle hub 122 prior to coupling the dispenser 292 to the needle hub 122 via the adaptor 290. In other examples, the adaptor 290 may be omitted and the dispenser 292 coupled directly to the needle hub. The PG unit 300 shown in FIG. 3 represents a removal state of the PG unit, following successful venipuncture. For example, a user can first grasp the catheter assembly 108 of FIG. 2 with the PG unit 300 of FIG. 3 and guide the tip of the needle 112 through the skin at a suitable insertion site to access a vein. Confirmation of proper vasculature access can be confirmed via primary blood flash, i.e., the presence of blood in the needle hub 122 and/or in the adaptor 290. The user can then retract the needle tip proximally of the catheter tube opening to observe secondary blood flash back, i.e., the presence of blood between the outer diameter of the needle 112 and the inner diameter of the catheter tube 110. In some instances, the needle can have a through notch formed through the wall of the needle shaft, near the needle tip, or a lengthwise groove by forming a depression lengthwise along the needle shaft. These alternatives allow flashback to be observed between the interior of the catheter tube and the exterior of the needle shaft and can be detected relatively quicker than blood flashback at the needle hub.

After successful vein access is confirmed, the dispenser 292 is activated by applying digital pressure on the guidewire 302 at the advancing or advancement platform 322 of the dispenser 292 and then sliding or feeding the guidewire 302 distally. The guidewire 302, such as the tip of the guidewire, may initially be disposed within the hollow needle 112, may only initially be disposed in the adaptor 290, or may only be initially deposed in the catheter hub 122. In some examples, the guidewire 302 can have a length with at least two distinct guidewire sections. For example, the guidewire 302 can have a distal guidewire section of a first diameter and a proximal guidewire section of a second diameter, which can be larger than the first diameter. The first and second diameters can be understood as outside diameters. A shoulder or a gradual taper can locate between the first and second guidewire sections.

Distal guidewire advancement can continue until the guidewire 302 has been fully extended within the vessel of the patient. In an example, the amount of extension of the guidewire into the vessel may be controlled by fixing the overall length of the guidewire 302 that can extend at the advancement platform 322. For example, the guidewire 302 may have two ends, a first end at the distal end and a second end at the proximal end of the guidewire. The length of the guidewire can be selected so that when the second end of the guidewire is moved to the advancement platform 322, it marks the full extended position of the guidewire. The user can then stop further advancement of the guidewire at that point. Optionally a physical stop or an indicator may be provided so that when the guidewire is advanced, the stop or the marker indicates how far the first end of the guidewire has extended distally beyond the needle tip 114, as further discussed below. Of course, the user does not have to fully extend the guidewire when using the catheter assembly with the PG 300 unit of the present invention.

In some examples, the relative lengths of the first and second guidewire sections can be selected to ensure appropriate dispensing of the first guidewire section out of the needle tip, such as to support the catheter during catheter insertion. In an example, as the guidewire is dispensed and the first diameter section of the guidewire exits the distal tip opening, the shoulder, the gradual taper, or the second diameter of the guidewire 302 can engage the needle at the crimp to stop distal advancement of the guidewire. In the present embodiment, the engagement between the change in profile of the needle and the shoulder of the guidewire will ensure a physical stop and prevent overextending the guidewire into the vein. A guidewire dispense length is defined from the point where the guidewire engages the crimp to the remote distal end of the guidewire. The guidewire dispense length can be selected to properly support the catheter tube during catheter insertion into the vein. In an example, the length of the guidewire from the far distal end to where the guidewire exits the needle tip, referred to as the support length, is approximately 50% of the length of the catheter tube. In an example, the support length is greater than 50% of the catheter length.

The catheter tube 110 (FIGS. 1 and 2) can now further be inserted into the vessel and be guided by the guidewire 302. As the catheter tube 110 and the catheter hub 130 move distally forward and the catheter tube guided by the guidewire into the vessel, the catheter hub 130 separates, or further separates, from the needle hub 122. The catheter tube 110 and catheter hub 130 can be advanced a sufficient amount over the guidewire 302 to ensure successful placement within the vessel. The PG unit 300 can then be removed or separated from the catheter tube and catheter hub. After removal, the PG unit 300 resembles the PG unit of FIG. 3, separated from the catheter hub and catheter tube.

Where the PG unit 300 of FIG. 3 is used with or as part of an extended dwell catheter assembly, such as the assembly 100 of FIG. 1, the method of using the assembly is similar to the process described immediately above except that when the catheter hub 130 is advanced to move the catheter tube 110 over the guidewire 302 (FIG. 3), the catheter hub or an attachment to the catheter hub can open the gates at the distal end of the housing 102 to allow separation of the catheter hub from the housing 102 (FIG. 1) or a user can activate levers, tabs, or buttons to split the housing into two or more housing sub-components to then allow the catheter hub to separate from the housing 102. The PG unit 300 can separate from the catheter hub at the same time, before, or after the housing separates from the catheter hub.

FIG. 3A is an enlarged view of the dispenser 292 and part of the adaptor 290 of FIG. 3. As shown, the dispenser 292 has a frame or body 306, a duct 308 engaged to the body 306, and a guidewire 302. The duct 308 can be a tubing length having two free ends and a lumen. The frame 306 has a sideway generally Y-shape configuration with a first branch 312, a second branch 314, and a third branch 316. The three branches 312, 314, 316 connect to one another at a focal or connected portion 318 of the frame or body 306 and each branch can have a respective free end 312a, 314a, 316a that extends away from the focal or connected portion 318. The free ends are spaced from one another and the frame 306 with the three branches 312, 314, 316 can be unitarily formed. In some examples, the third branch 316 can be omitted and the second end of the tubing can loop around and be held to itself via one or more tubing clips, as further discussed below. The embodiment without the third branch 316 may be used with any of the adaptors and any of the PG units discussed herein.

As shown, the first branch 312 has a free end 312a that generally points in the distal direction, the second branch 314 has a free end 314a that generally points in the proximal direction, and the third branch 316 has a free end 316a that generally points radially of a lengthwise axis defined by the first and second free ends 312a, 314a, and the free end of the third branch optionally also points proximally in addition to pointing radially. In an embodiment, each branch of the frame 306 is elongated and can have surface contours and/or arcuate bends. For example, the first branch 312 can extend distally from the connected portion 318 and can have a reduced thickness section defining a guide platform 322, also referred to as an advancing or advancement platform. The reduced section can be formed by providing a surface 322a that resembles a section of an arc, which has sections of a relatively larger width or thickness on either side of the reduced section.

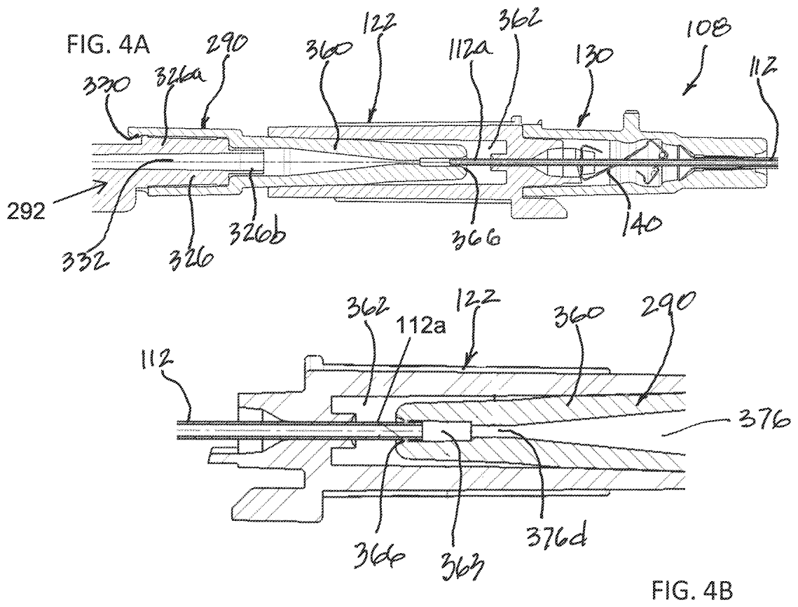

The first branch 312 can have a connection tip 326 at a distal end thereof. The connection tip 326 resembles a reducer and reduces down to act as a slip-on tip or nipple, for connecting to the open proximal end 290a of the adapter 290. With further reference to FIG. 4A, which shows part of the dispenser 292, adaptor 290, and catheter assembly 108 in a cross-sectional view, the connection tip 326 has a first tip section 326a and a smaller diameter second tip section 326b. The size and shape of the connection tip 326, including the first and second tip sections 326a, 326b, are configured to mate with the female bore of the adaptor 290. In an example, the engagement between the two is a taper interference fit, similar to a Luer fit but not necessarily formed to the Luer ISO standard. In yet other examples, the adaptor 290 and the connection tip 326 of the dispenser 292 can be provided with a detent or detent-like engagement 330. As shown, the connection tip 326 and the receiving bore of the adaptor can include a raised lip or shoulder and a corresponding recess to form a snap fit. As shown, the lip 330 can grip a flange or shoulder on the tip of the dispenser 292. In still other examples, rather than a connection tip 326 with different tip sections, such as different tip diameters, the connection tip can embody a single taper for engaging a corresponding taper of the adaptor, similar to a Luer taper but with non-standard ISO sizes. In still other examples, the tip 360 of the adapter 290 engages the opening of the needle hub 122 in a Luer fit. The connection tip 326 of the dispenser 292 can also engage the proximal opening of the adapter 290 in a Luer fit. In some examples, the connection tip 326 of the dispenser is sized and shaped to engage the needle hub directly without the adaptor. That is, in some embodiments, a guidewire dispenser can have a connection tip that connects directly to the needle hub and wherein the body of the dispenser can have two or three branches. For the dispenser embodiment 292 without an adaptor 290, the connection tip 326 can be extended to ensure alignment with the needle proximal opening to facilitate guiding the guidewire into the needle lumen. In other examples, the body of the dispenser 292 can be unitarily formed with the adaptor 290.

The first branch 312 can have a guide passage 332 extending axially through the first branch and through the connection tip 326. The guide passage 332 is provided for passing the guidewire 302 therethrough and into the adaptor 290, and into and out of the needle 112 during use for guiding the catheter tube, as previously discussed. For example, the guidewire 302 has two ends, called a first end or distal end and a second end or proximal end. In a ready to use position, the first end can extend through the guide passage 332, out the connection tip 326 and into the interior of the adaptor 290. In an example, the first end or distal end of the guidewire 302 does not enter the needle lumen in the catheter assembly ready to use position to provide flow space for primary blood flashback during needle puncture, as further discussed below.

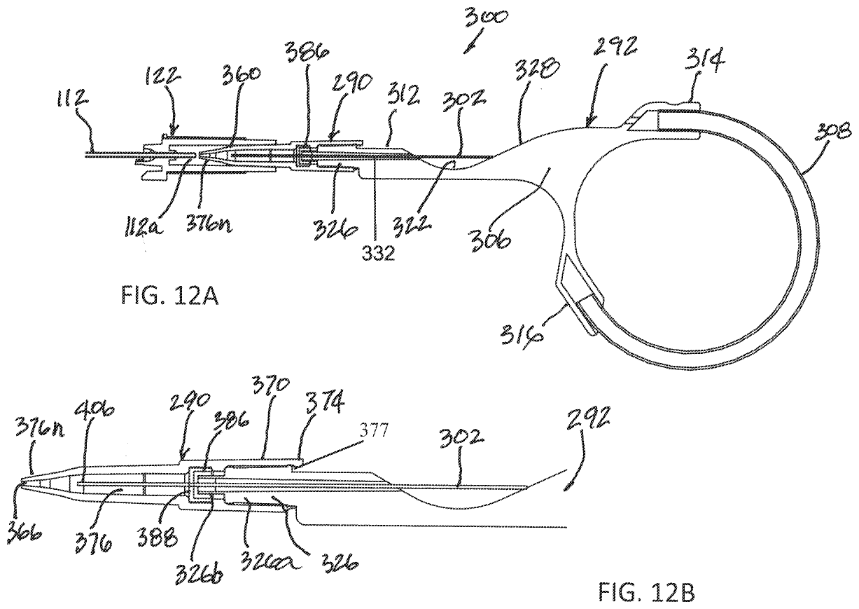

With reference again to FIG. 3A in addition to FIG. 12A, the dispenser 292 has a sloped section 328 that inclines in the distal to proximal direction towards the second branch 314. A guide bracket 334 is located at the proximal end of the second branch 314. The guide bracket 334 has a body 338 that is generally cuboid in shape, such as resembling a rectangular prism, with a slanted or tapered distal end or distal side 336, which is sloped or tapered. In some examples, the guide bracket 334 has a different cross-sectional shape, such as round or polygonal and the distal side can be more vertically aligned.

A guide passage is provided axially through the body 338 of the guide bracket 334 to accommodate the guidewire 302. Some of the guidewire 302 resides in the duct attached to the guide bracket 334. Elevation-wise, the distal end or side 336 of the guide bracket 334 seats higher than the entrance to the connection tip 326 at the end of the first branch 312. This elevation difference can facilitate guidewire dispensing by allowing the dispenser 292 to be gripped while using the thumb to slide the guidewire 302 in the distal direction, as further discussed below. However, the angle of the guidewire extending between the guide bracket 334 at the second branch and the guide tip 326 at the first branch can vary while still providing the dispenser with an ergonomic shape, such as the dispenser of FIG. 12B. For example, the distal end or side 336 can be at the same elevation as the entrance to the connection tip 326.

The guide bracket 334 at the second branch 314 can be provided with a socket 340 for receiving a first end 308a of the duct 308. In an example, the guide bracket 334 can have a plurality of walls defining a round socket 340 therein. In an example, the duct 308 can embody a round tubing section and the first end 308a of the tubing can engage the round socket 340 of the guide bracket 334, such as in a friction fit, an interference fit, or a snap fit. Using the duct 308 with the frame 306 of the dispenser 292 is preferred but not required as the guidewire 302 can simply extend out the second branch 314 and dangle proximally of the second branch 314 if the duct 308 is not used, which is not preferred. As incorporated, the duct 308 receives part of the guidewire 302 therein to prevent kinking or bending of the guidewire. In still alternative examples, a duct having a shorter length than shown can be incorporated and the second end of the duct can dangle in the air and not attach to the socket 342 at the third branch 316. The two sockets 340, 342 can have any internal bore shape provided the shape can grip or couple to ends of a tubing.

In an example, the second end 308b of the duct 308 and the guidewire 302 both dangle from the second branch 314, with the second end 308b of the tubing unsupported. In other words, the guidewire dispenser can be without the third branch, i.e., the third branch can be omitted. Alternatively and as shown, the duct 308 has a curved body to curve around so that the second end 308b of the duct 308 projects into the socket 342 of the support bracket 349 of the third branch 316, in a friction fit, interference fit, or a snap fit. The socket 342 can be round and formed by wall surfaces of the support bracket. The duct 308 in the embodiment shown is therefore supported at both the first end 308a and the second end 308b by the guide bracket 334 and the support bracket 349, respectively, and the mid-section 308c between the two ends suspended. The support bracket 349 can be practiced without a guidewire passage through the third branch 316 should the length of the guidewire be sufficiently short and not extend past the opening at the second end 308b of the duct. However, a guidewire passage can be incorporated through the support bracket 349 at the third branch 316 as necessary to accommodate the length of the guidewire.

The support bracket 349 at the third branch 316 is generally cuboid in shape, such as resembling a rectangular prism, with a slanted or tapered remote end or distal side 346, which is sloped or tapered. In some examples, the support bracket 349 has a different cross-sectional shape, such as round or polygonal and the distal side can be more vertically aligned.

The body 306 of the dispenser is provided with a curved or arcuate edge or surface 350 between the third branch 316 and the first branch 312. Similarly, the body 306 is provided with a curved or arcuate edge or surface 352 between the third branch 316 and the second branch 314. The curved edge 352 between the third branch 316 and the second branch 314 of the frame 306 and the curved duct 308 form a generally round structure having a hollow space 356 or opening for receiving or accommodating the guidewire 302 and for gripping. For example, the curved edge 350 between the third branch 316 and the first branch 312 provides a gripping surface for the user. As shown, the duct 308 and the frame 306 formed a closed loop. In particular, the duct 308 and the curved edge surface 352 of the frame 306 define a generally round structure. Obviously, while the curved edges 350, 352 of the frame 306 are preferred for the reasons noted, they can be modified to be less curved or even have straight sections and still provide the general functions described without also defining a round structure.

During use, the user, in a one-handed use procedure, can grip the dispenser by placing the middle finger, ring finger, and pinky finger into the hollow interior 356 of the dispenser and up against the curved edge 352. The index finger can grip around the curved edge 350 between the third branch 316 and the first branch 312, opposing the inner curved edge 352. The thumb can then be placed over the upper side of the dispenser 292, over the sloped section 328, and above the guidewire 302 and the guide platform 322. To dispense or activate the guidewire 302, the user uses the thumb to press the guidewire 302 against the surface 322a at the guide platform 322 with both a downward force against the surface 322a and a distally directed force to move the guidewire 302 in the distal direction. This will move the first end of the guidewire through the needle and out the distal end thereof to help guide the catheter tube. Total guidewire movement out the needle tip can be controlled by controlling or sizing the total length of the guidewire so that when the second end of the guidewire is pushed into connection tip 326 at the first branch 312, no further guidewire is available to dispense. In other examples, the second end of the guidewire 302, or some part of the guidewire, can be provided with a catch, such as a hook, an enlarged section, such as a larger diameter section at the proximal guidewire section, a knot, etc., that catches on the frame 306 of the dispenser 292 or catches the crimp area of the needle after the guidewire 302 has dispensed some finite amount to prevent the guidewire from further distal movement out the connection tip. For example, and as previously discussed, the guidewire can have at least two wire diameter sections with a shoulder or transition between the two diameter sections, which can be configured to engage the crimp or change in profile on the needle to stop distal advancement of the guidewire.

In some examples, a crimp can be incorporated with the needle for engaging an opening on the needle guard, when withdrawing the needle following successful venipuncture. When the crimp is used, the crimp can define a relatively smaller internal opening in the needle lumen than the nominal inside diameter of the needle lumen, because of actual crimping of the needle to create the crimp. The relatively smaller internal opening or lumen at the crimp can be used as a limiter, physical barrier, or limiting means to limit distal advancement of the guidewire. For example, the guidewire can include a bead or an enlarged section at a predetermined point along the guidewire between the two ends, i.e., between a first end at the distal end and a second end at the proximal end.

When a bead or an enlarged section on the guidewire passes through the needle, after the distal end of the guidewire advances out through the needle tip, the bead or the enlarged section is physically restricted from passing distally through the relatively smaller internal opening at the crimp, thereby stopping any further advancement of the first end of the guidewire out of the needle tip. The location of the bead or the enlarged section relative to the first end of the guidewire can be selected to control the length of the first end that travels out the needle tip, and therefore enters the vasculature of the patient when the guidewire is in use. In an example, the first end travels outside the needle tip an amount that is approximately 50% of the length of the catheter. The length of travel can be greater than 50% of the length of the catheter. However, the length can be less than 50% of the length of the catheter, such as 20% to 50% of the length of the catheter.

In some examples, the guidewire is provided with at least two different guidewire sections with two different outside diameter dimensions. For example, the distal guidewire section can have a first OD and the proximal guidewire section can have a second OD and wherein the second OD is larger than the first OD. The first OD can be sufficiently small to pass through the needle lumen at the crimp but not the second OD, which has a larger cross-section than the pass-through dimension of the lumen at the crimp. The length of the distal guidewire section can be selected to represent the length of the guidewire that will pass through the patient's vasculature or vein when the guidewire is stopped at the crimp. A shoulder can be provided at the interface between the two guidewire sections and the shoulder can abut the crimp to serve as a stop. In other examples, a gradual transition can be provided between the two guidewire sections. The shoulder, the gradual transition, or the second OD of the guidewire can be sized to abut the crimp section of the needle when dispensing the guidewire out through the needle tip to function as a stop. Thus, the assembly can be provided with a safe stop to stop further advancement of the guidewire into the vein.

In an example, a guidewire with at least two guidewire sections with two different guidewire OD sizes can be used without the guidewire dispenser housing. In this variation, an adaptor, such as one of the adaptors discussed herein, can be connected to the needle hub. The user can then hold the guidewire directly without the guidewire dispenser housing to advance the guidewire through the adaptor and into the needle lumen. Advancement of the guidewire can stop when a shoulder, a gradual transition, or the second OD of the guidewire catches the needle at the crimp. In some examples, the guidewire, the guidewire dispenser, or both the guidewire and the guidewire dispenser can incorporate a code or coding to indicate the guidewire gauge size. The code or coding can be a color, a numerical code, an alpha code, an alpha numeric code, or combinations thereof.

With reference now to FIGS. 4A and 4B, which is an enlarged view of the connection between the adaptor 290 and the needle hub 122 of FIG. 3A, use and assembly of the various components will be described. As shown, the tip 360 of the adaptor 290 projects into the interior space 362 of the needle hub 122 and the distal opening 366 at the distal end 367 of the tip 360 is located over the proximal end 112a of the needle 112, which has a blunt end. In an example, the bore or cannula channel 363 at the distal opening 366 (FIG. 4B) of the tip 360 is sized to have a size-on-size fit with the needle proximal end 112a with a loose fit, such as having a small clearance between the two, being optionally employable. A flared inlet or entrance can be provided at the distal opening 366 of the tip 360. The flared inlet at the distal opening 366 allows the tip 360 to readily slide over the needle to receive the proximal end 112a into the bore of the tip 360 and helps to compensate potential slight misalignment.

The tip 360 can be provided with a modified Luer taper to engage the needle hub 122 in a tapered fit at the proximal opening of the needle hub. The tip 360 can be elongated so that the distal end of the tip 360 can reach and project over the needle proximal end 112a to receive the needle inside the bore 363 of the tip 360.

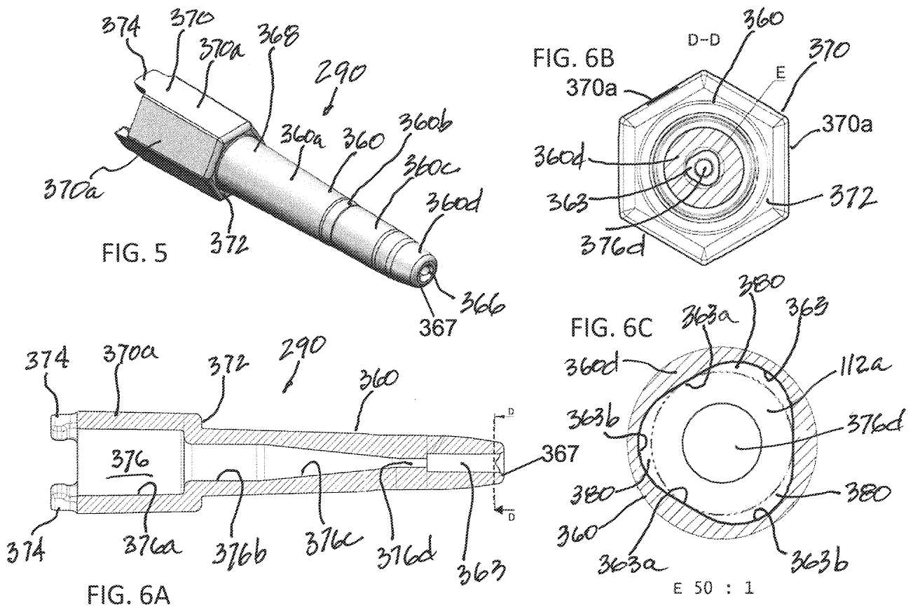

FIG. 5 is a perspective view of an adaptor 290 provided in accordance with aspects of the invention, which is similar to adaptors discussed elsewhere herein. The adaptor 290 in the present embodiment has a body 368 comprising a base 370 and a tip 360 with a shoulder 372 located between the two. Exteriorly, the base 370 is provided with a plurality of slats or sidewalls 370a that are inter-connected and angled relative one another. At the end of at least one of the slats 370a, a flange or tab 374 is provided. The flange or tab 374 can be continuously formed with the slat 370a from which it extends. The flange or tab 374 can have at least three unobstructed side edges. In other examples, the flange or tab 374 can be rounded, such as being partially circular, or can have more than three edges. There can be more than one flange or tab 374 incorporated at the base 370. In a preferred embodiment, at least two spaced apart flanges or tabs 374 are incorporated at the base 370. When two spaced apart flanges are incorporated, the tip of the dispenser placed therebetween deflects the two flanges and engages them in a detent engagement. For example, the tip of the dispenser 292 can have a shoulder or a lip 377 (FIG. 12B) and the two or more flanges or tabs 374 can grip the shoulder or lip to aid in engaging the dispenser to the adaptor. Thus, broadly speaking, the dispenser can comprise a first structure 374 that engages a second structure 377 on the dispenser.

The tip 360 can be generally frustoconical in shape with one or more reduced tip sections. For example, the tip 360 can have a first tip section 360a with a first size, a second tip section 360b with a second size, a third tip section 360c with a third size, and a fourth tip section 360d with a fourth size. In other examples, there can be fewer or more than the number of sections shown depending on the fit desired with the interior of the catheter hub 122 and the reach of the tip inside the needle hub to either surround or be adjacent the needle proximal end. In other examples, the tip 360 can have a continuous frustoconical shape without distinct reduced sections, such as section 360b. However, the tip 360 of the present adaptor 290, as well as other adaptors discussed herein, can have other bore shapes, other than a frustoconical shape, and still encompasses aspects of the invention.

FIG. 6A shows a lengthwise cross-sectional side view of the adaptor 290 of FIG. 5. FIG. 6B shows an end-view of the adaptor 290 taken along line D-D and FIG. 6C is an enlarged view of the central section E of FIG. 6B. With reference initially to FIG. 6A, the two flanges or tabs 374 are opposed along a lengthwise axis and each comprising an interior surface having a lip or a surface engagement feature. The gap distance between the two tabs 374 can be relatively smaller than the inside diameter of the base 370. This relatively smaller dimension between the two flanges or tabs 374 can be selected to engage a lip or corresponding surface on the connection tip of a guidewire dispenser, as shown in FIG. 4A.

Internally, the adaptor 290 can have a bore 376 that is open so that the proximal end is in fluid communication with the distal end of the adaptor. In an example, the bore 376 has different bore sections with different bore diameters. The bore 376 extends through the length of the adaptor 290, including at the base 370 and defines a base bore. The different bore diameter sections can be selected or sized to fit with selected connection tip of a guidewire dispenser. With reference again to FIG. 4A in addition to FIG. 6A, the bore 376 can have a first bore section 376a having a first diameter for coupling to the first tip section 326a of the dispenser and a second bore section 376b having a second diameter for coupling to the second tip section 326b of the dispenser. The various sections of the bore 376 can also be tapered to match corresponding taper on the tip of the dispenser. Distal of the second bore section 376b, a third bore section 376c is provided. The third bore section 376c can have a frustoconical shape that necks down to a guidewire passage 376d and then expands in diameter to the cannula channel 363, which has a larger cross-sectional space than the guidewire passage as the needle has a larger outside diameter than the guidewire diameter. At the distal end of the adaptor 290, a flare inlet is provided at the distal opening 366. The f tapered end most edge of the flared inlet is provided to facilitate placement of the cannula channel 363 over the blunt proximal end of the needle, as previously discussed. In an example, the first bore section 376a has a larger inside diameter than the inside diameter of the second bore section 376b, which has a larger inside diameter than the third bore section 376c, which has a larger inside diameter than the guidewire passage 376d, which has a smaller inside diameter than the cannula channel 363. Thus, the tip has an inside diameter at the guidewire passage 376d that is smaller than the two adjacent bore sections, i.e., the third bore section 376c and the cannula channel 363.

FIG. 6B shows a cross-sectional end view of the adaptor 290 taken along line D-D of FIG. 6A. From the exterior most component to the interior most component of the adaptor 290 of FIG. 6B, the base 370 can be seen with six slats or sidewalls 370a, the shoulder 372, the tip 360, the cross-section of the fourth tip section 360d, i.e., the wall of the fourth tip section, the cannula channel 363, and the guidewire passage 376d.

With further reference to FIG. 6C in addition to FIG. 6B, the cannula channel 363 has a non-circular interior space defined by three generally straight sides 363a connected to one another by arcuate connecting sections 363b. If the proximal end 112a of a needle 112 is placed into the cannula passage 363, the needle proximal end 112a would resemble the ring-shaped structure shown in dashed lines in FIG. 6C and would contact or be in close contact with the three generally straight sides 363a of the cannula channel 363. The needle bore would align centrally with the guidewire passage 376d shown. The contact between the exterior of the needle 112 and the three generally straight sides 363a can generate a slight interference fit to ensure a firm connection between the cannula channel 363 and the needle 112. Each arcuate connection section 363b and the needle exterior define a bypass gap 380 at the cannula channel 363. As shown, the tip 360 of the adaptor 290 and the needle at the cannula channel 363 define three bypass gaps 380. In other examples, there can be more than three straight sides and corresponding number of arcuate connection sections. For example, there can be four straight sides 363a connected by four arcuate connecting sections 363b. In still other examples, the cannula channel 363 contour can have different shapes than as shown. For example, the three straight lines, where they contact the needle, can be slightly arcuate, with a different curvature than the arc of the needle outer diameter.

With reference again to FIG. 4B in addition to FIG. 6C, in use during initial needle puncture, a guidewire 302 (FIG. 3A) occupies the guidewire passage 376d at the tip 360 of the adaptor 290 but the guidewire first end does not project into the needle lumen, at the needle proximal end. Thus, when primary blood flashback flows proximally out of the needle proximal end, the blood first flows into the cannula channel 363 but cannot flow proximally into the bore 376 at the base 370. This is because the guidewire 302 (FIG. 3A) occupies the guidewire passage 376d and the clearance between the guidewire 302 and the bore diameter at the guidewire passage 376d may be too restrictive for blood to flow thereacross.

However, because the bypass gaps 380 can be sized for blood flow, such as by providing a sufficient flow passage or clearance at each bypass for a given fluid viscosity, blood entering the cannula channel 363 can flow out through the bypass gaps 380 and into the interior space 362 (FIG. 4B) of the needle hub 122. A practitioner can thus detect primary blood flashback at the interior space 362 of the needle hub 122 after blood flashback flows into the cannula channel 363 of the adaptor 290 and then reverses flow and flow out through the bypass gaps 380 and into the interior space 362 of the needle hub 122 to be detected visually through the wall surfaces of the needle hub, such as by perceiving the blood red color within the interior space 362.

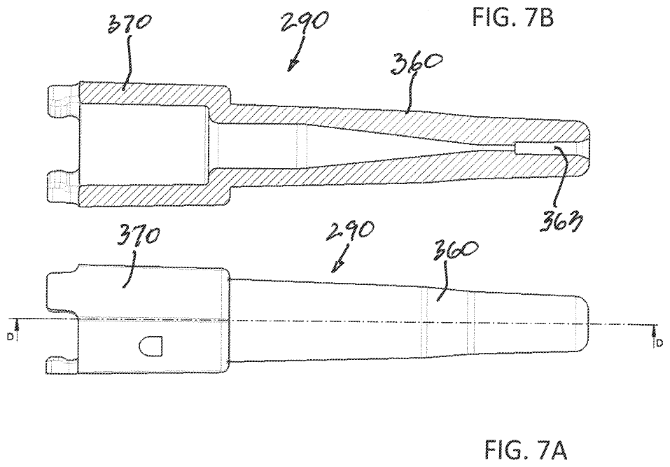

FIG. 7A is a side elevation view of the adaptor 290 of FIG. 5 and FIG. 7B is a cross-sectional side view thereof taken along line D-D of FIG. 7A, which is the same as the cross-sectional view of FIG. 6A.

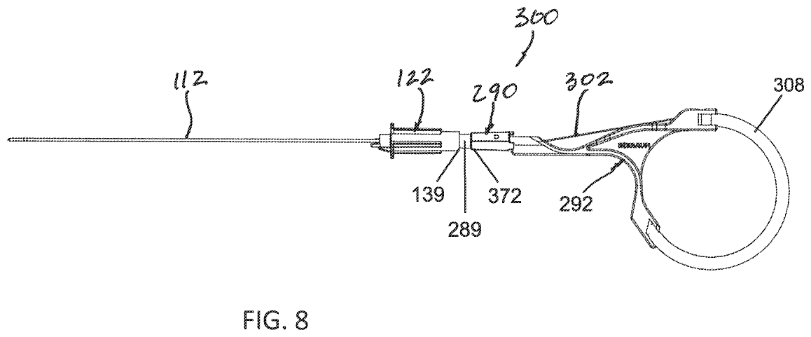

FIG. 8 is a side elevation view of the PG unit 300 of FIG. 3, showing a needle hub 122 having a needle 112, an adaptor 290, and a guidewire dispenser 292 having a guidewire 302. In the example shown, the proximal end edge 139 of the needle hub 122 is spaced from the shoulder 372 on the adaptor 290 by a gap 289. The shoulder 372 being located between the tip and the base of the adaptor (FIG. 6A). The gap 289 exposes that portion of the tip between the needle hub and the base to the atmosphere.

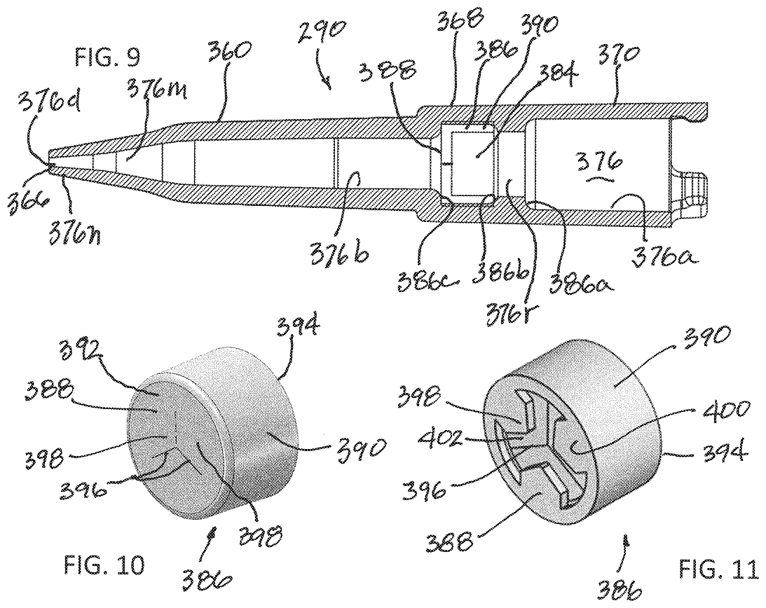

FIG. 9 is a cross-sectional side view of an adaptor 290 provided in accordance with further aspects of the invention, which is similar to the adaptor 290 of FIGS. 5 and 6A-6C with some variations. Like the earlier described adaptor 290, the present adaptor has a body 368 comprising a base 370 and a tip 360. The base 370 can similarly have a plurality of slats or sidewalls 370a joined along respective edges. One or more flanges or tabs 374 can extend proximally of the base 370 with each flange or tab 374 optionally incorporating engaging features for engaging the guidewire dispenser 292, as previously discussed.

The tip 360 of the present adaptor 290 can be provided with different exterior tip sections as well as different interior tip sections, as previously discussed. In the present embodiment, the distal end most of the tip 360 has a bore that is provided with a shaped internal bore 376m, resembling a frustoconical shaped section. The shaped internal bore 376m is distal of a first bore section 376a and second bore section 376b and has a reduced tip defining a guidewire passage 376d. The guidewire passage 376d is sized and shaped to accommodate a guidewire passing therethrough but is otherwise too small to accommodate a needle proximal end. Thus, compared to the adaptor 290 of FIG. 6A, the adaptor 290 of FIG. 9 does not incorporate a cannula channel 363 (FIG. 6A) distal of the guidewire passage 376d for receiving a needle proximal end. In use, similar to the configuration shown in FIG. 4B and as further discussed below, the distal most tip 376n of the adaptor 290 can be spaced from the proximal end of the needle and any primary blood flashback flowing through the needle lumen can simply flow directly into the interior space 362 of the needle hub 122 (FIG. 4B).

In an alternative embodiment, the present adaptor 290 can be lengthened and incorporate a structure distal of the guidewire passage 376d to define a cannula channel, similar to the adaptor 290 of FIG. 6A and the cannula channel 363 incorporated therewith. If incorporated, the adaptor 290 of FIG. 9 can receive the proximal end of a needle in the cannula channel when the adaptor 290 is engaged to the needle hub, similar to that shown in FIG. 4B. Blood flashback into the cannula channel of the modified adaptor 290 of FIG. 9 with a cannula channel can therefore reverse and flow out of bypass gaps at the modified tip, as discussed with reference to FIG. 6C.

The base 370 of the present adaptor 290 is provided with a valve chamber 384 having a valve 386 located therein. In an example, the base 370 is provided with internal shoulders 386a, 386b, 386c to form varying internal chamber sizes or sections, including a first bore section or chamber 376a, a valve chamber 384, and a reduced chamber 376r located between the first bore section 376a and the valve chamber 384. In an example, the first bore chamber 376a is configured to receive a connection tip of a guidewire dispenser, similar to the embodiment of FIG. 4A, and the reduced chamber 376r is configured to accommodate a reduced tip section of the connection tip of the dispenser, as further discussed below. In some examples, the base 370 can be a continuous or can have a generally cylindrical structure without distinct slats and edges formed between the slats.

A valve 386 can be placed in the valve chamber 384 and held therein by the distal shoulder 386c and the proximal shoulder 386b. In an example, the valve 386 can have a valve disc 388 and a valve skirt 390 extending from the valve disc. The valve disc 388 can have a wall with a distal surface 392 of the wall pressing or contacting the distal shoulder 386c and the proximal end 394 of the valve skirt 390 can press or contact the proximal shoulder 386b to position the valve therebetween. In some examples, the valve 386 can be provided with a valve disc without a skirt section depending therefrom and the valve chamber 384 can be modified to receive the modified valve.

In an example, the valve 386 shown in FIG. 9 can resemble the valve 386 shown in FIG. 10, shown in front perspective view. Said differently, the valve 386 of FIG. 10 can be practiced with the adaptor 290 of FIG. 9. With reference to FIG. 10, the valve 386 of the present embodiment can have a valve disc 388 and an integrated skirt 390. In the example shown, the valve disc 388 and the skirt section 390 are unitarily formed. The skirt section 390 can be viewed as a generally cylindrical length having an open proximal end, having a proximal most edge 394. In some examples, the skirt 390 can be omitted and only the valve disc 388 is positioned in the adaptor. The valve disc 388 is shown with a plurality of slits or valve disc slit portions 396 defining a plurality of valve flaps 398. For example, the valve can have three slits defining three flaps. However, the valve can be practiced with one slit defining two flap sections or more than three slits and three flaps. The three slits can meet at a focal or central point. The slits are preferably equal in length but not required to be equal and the slits stop short of the outer diameter of the valve disc. In the present embodiment, the wall thickness of the valve disc 388, in the axial direction, is generally constant, without varying surface features. However, distinct surface features can be incorporated, as shown in FIG. 11. In some examples, a pierceable septum is used instead of a valve.

In some examples, the valve skirt 390 can have a generally constant outside diameter (OD). The valve skirt 390 can have a generally constant thickness so that the inside diameter of the skirt can be generally constant. In alternative embodiments, the thickness can vary so that the outside diameter of the skirt section can be constant while the inside diameter can vary.

FIG. 11 shows an alternative valve 386 that is usable with the adaptor 290 of FIG. 9. As shown and similar to the valve 386 of FIG. 10, the valve 386 of FIG. 11 has a valve disc 388 and a valve skirt 390 depending therefrom, similar to the valve of FIG. 10. The valve disc 388 is shown with a plurality of slits or valve disc slit 396 defining a plurality of valve flaps 398. In the present embodiment, the wall thickness of the valve disc 388, in the axial direction, has a distinct first portion 400 and second portion 402. The first portion 400 can have a first thickness and the second portion 402 can have a second thickness and wherein the first thickness can be greater than the second thickness, measured orthogonal to a medial plane passing through the diameter of the valve. The second portion 402 with the second thickness can have a substantially constant thickness but can optionally include varying thicknesses along a cross-section of the valve, at the second portion 402.

In an example, the second portion 402 can be formed by recessing the distally facing surface 392 of the valve disc 388, the proximally facing surface of the valve opposing the distally facing surface, or both the distally and proximally facing surfaces of the valve disc 388 while the first portion retains substantially the full width or thickness of the valve between the proximally and distally facing surfaces. In an example, the recesses at the second portion can embody undercuts formed into the valve.

The surface appearance between the first and second portions 400, 402 can resemble a three-leaf clover. The three-leaf clover can be present on the distally facing surface, the proximally facing surface, or both surfaces of the valve disc. In other examples, the surface appearance of the proximally and/or distally facing surfaces can have varying contours so that the three-leaf clover can have contours of varying curves, lines, and edges. The surface features can facilitate flexing and scaling of the valve at the slits. The valve 386 can alternatively embody a different shape. For example, the length of the skirt can be shorter than as shown. The number of slits can be more than or less than three. The skirt can optionally incorporate air vents for to enable air venting across the exterior of the skirt section but not large enough for blood flow thereacross.

FIG. 12A shows a PG unit 300 having a dispenser 292, an adaptor 290, and a needle hub 122 having a needle 112 extending therefrom, with the needle only partially shown. The PG unit 300 of the present embodiment is similar to the PG unit 300 of FIG. 3 with a few exceptions. In the present embodiment, the dispenser 292 can embody the dispenser of FIG. 9, which can have a valve 386 located therein. The valve can have a valve disc and a valve skirt. For example, the valve 386 of FIG. 12A can embodiment the valve 386 of FIG. 10 or FIG. 11. Further, while the distal most tip 376n of the dispenser is spaced from the proximal end 112a of the needle 112, the tip 360 can include a cannula channel 363 and the needle proximal end 112a can be located in the cannula channel, similar to the embodiment of FIG. 4B.

The dispenser 292 of FIG. 12A is similar to the dispenser of FIGS. 3 and 3A and includes three branches 312, 314, 316. However, in the present embodiment, the frame or body 306 has been modified so that the guidewire 302 extends generally horizontally and generally coaxial with the needle through the guide platform 302 of the frame. The frame 306 can be provided with a guide passage at the sloped section 328 that inclines towards the guide bracket 334 at the second branch 334. This arrangement lowers the angle of insertion of the guidewire 302 as the guidewire extends towards the connection tip 326 of the dispenser and into the adaptor 290.

FIG. 12B is an enlarged view of the PG unit 300 without the needle hub 122 for clarity. As shown, the connection tip 326 of the dispenser 292 projects into the receiving space or bore 326 at the base 370 of the adaptor 290 and the one or more tabs 374 engage corresponding detent surfaces on the dispenser 292, such as a shoulder, a lip, or a flange, to maintain a firm engagement between the adaptor and the dispenser. Optionally the engagement can be an interference fit without use of the flanges or tabs.

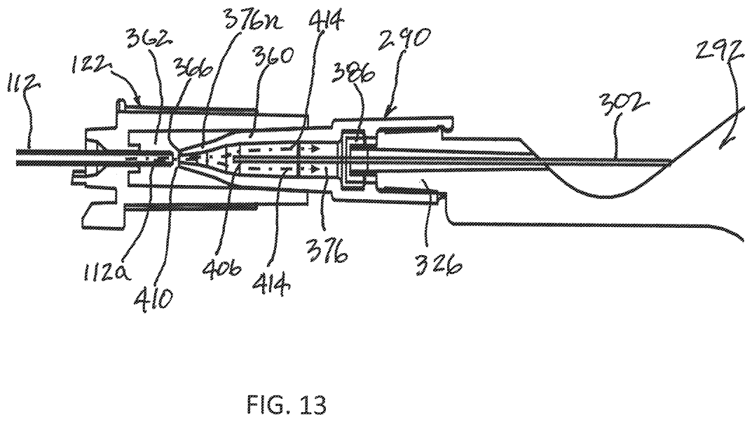

As shown, the distal tip section 326b of the dispenser 292 is located in the interior space defined by the skirt 390 of the valve 386 but does not push through the valve disc to deflect the valve flaps. In an example, the distal tip section 326 of the dispenser 292 is spaced from the proximally facing surface of the valve disc 388. The guidewire 302 extends through the slits of the valve disc such that the first end 406 of the guidewire 302 is located in the bore 376 of the adaptor and recessed from the distal most tip 376n of the adaptor. The location of the first end 406 as shown can be practiced in the ready to use position of the catheter assembly, as further discussed below with reference to FIG. 13.

FIG. 13 is an enlarged view showing a catheter assembly or needle assembly having an adaptor 290 and a dispenser 292 in accordance with aspects of the invention, shown without a catheter hub 130 (FIG. 2) for clarity. Optionally, an outer housing 102 (FIG. 1) can also be used but not shown for clarity. As shown, the tip 386 of the adaptor 290 is positioned in the interior space 362 of the needle hub 122 and the connecting tip 326 of the dispenser 292 is located inside the bore 376 at the base of the adaptor 290. The distal most tip 376n of the adaptor 290 is spaced from the needle proximal end 112a such that a gap 410 is provided therebetween. The first end 406 of the guidewire 302 is located in the bore of the adaptor 290 and recessed from the distal most tip 376n of the adaptor. The adaptor 290 has a valve 386 located therein, as shown and described with reference to FIGS. 12A and 12B.

Following initial vascular access, blood flows in the proximal direction through the lumen of the needle 112 and out the proximal opening of the needle. Because of the gap 410 between the needle and the tip 360 of the adaptor 290, some of the blood spills into the interior space 362 of the needle hub 122, which can then be detected visually through the walls of the needle hub as primary blood flashback. Some blood may also flow through the distal opening 366 at the tip of the adaptor 290, which has a guidewire passage 376d (FIG. 9). Blood flow 414 through the bore 376 of the adaptor 290 can be restricted or stopped by the valve 386 located in the valve chamber inside the adaptor 290. This way, a practitioner can take appropriate time to place the guidewire into the vein and then slide the catheter tube over the guidewire to place the catheter tube deep into the vein without feeling rushed due to the outpouring of blood out through openings of the adaptor and/or of a dispenser if no seal 386 is incorporated inside the adaptor. Following successful venipuncture, the PG unit 300, which includes the needle hub 122, adaptor 290, and dispenser 292, are removed from the catheter hub and catheter tube. The components can be removed as a single integrated unit or can be removed individually.

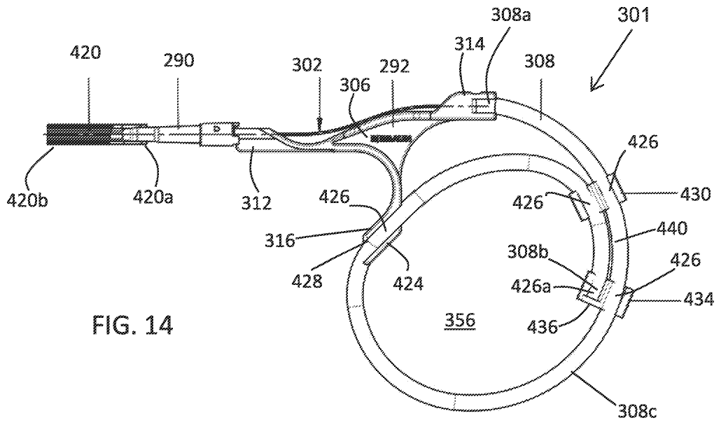

With reference now to FIG. 14, a guiding unit 301 comprising an adaptor 290 and a guidewire dispenser 292 is shown. The guiding unit 301 does not include a needle and needle hub. In the present embodiment, the guidewire dispenser 292 is similar to guidewire dispensers discussed elsewhere with a few changes. The dispenser 292 is shown with a guidewire 302 and a duct 308 and connected to an adaptor 290, which can be one of the adaptors discussed elsewhere herein. A protective cap 420 is provided at the tip of the adaptor 290. The assembly shown in FIG. 14 can be provided in a shipping package, such as blister pack or a thermoplastic pouch, and be commercially available as a unit as shown. Alternatively, the dispenser can be one of the dispensers discussed elsewhere herein.

The protective cap 420 can have a first end 420a and a second end 420b and formed as a thermoplastic protective sleeve. The first end 420a can have a bore that is sized and shaped to snap fit over the tip of the adaptor 290. Optional detent engagement and/or friction fit between the two can be practiced. The second end 420b can be a closed end or can have an opening. The protective cap 420 can be removed from the adaptor 290 and disposed of prior to using the adaptor and dispenser unit.

The dispenser 292 of FIG. 14 is similar to the dispenser of FIGS. 3, 3A, 12A, and 12B and includes three branches 312, 314, 316. However, in the present embodiment, the frame or body 306 has been modified so that third branch 316 now has a C-channel 424 rather than a socket 342 (FIG. 3A). Further, the third branch 316 can point away from the second branch 314. The C-channel 424, which can be called a retaining clip, can be a pass-through clip at the third branch 316 having a slot 426. That is, the duct 308 does not end at the third branch 316 but is routed through the clip so that the second end 308b is away or remote from the C-channel. The duct 308 can snap fit into the clip 424 by placing the duct over the slot 426 and then pressing the duct through the slot to be seated within the C-channel. Less optimally, the second end 308b of the duct 308 can be routed through the C-channel 424 by inserting into the end opening 428 and then pushing the second end 308b through.

An intermediate retaining clip 430 is provided to hold parts of the mid-section 308c of the duct together. In the example shown, the intermediate retaining clip 430 has an integrated or a unitarily formed body with two slots 426, 426 for receiving two different sections of the duct 308. The intermediate retaining clip 430 can be viewed as two C-channels that are formed side-by-side, with a common center side. Both C-channels are pass through in that the tubing does not terminate at any of the C-channels.

A second intermediate clip 430 (not shown) can be used to support additional sections of the tubing or duct 308. For example, the second intermediate clip 430 can be placed adjacent the first intermediate clip 430, either in contact with or spaced from the first intermediate clip 430, to support the duct near the second end 308b, with the second end 308b passing through the second intermediate clip 430.

Alternatively or additionally, an end clip 434, which can broadly be called a retaining clip, can be provided to support the duct or tubing 308, and particularly the second end 308b of the duct. The end clip 434 can be viewed as a C-channel and a modified C-channel that are formed side-by-side, with a common center side. The end clip 434 has a C-channel that has a pass-through slot 426 and a closed slot 426b in which an end wall 436 is provided. The end wall 436 prevents the second end 308b from passing through the modified C-channel 426a.

By using one or more intermediate clips 430 and optionally with an end-clip 434 in combination with the pass-through C-channel 424 at the third branch 316, the tubing 308 can be coiled to have an overlapping section or sections 440. That is, the tubing 308 can be coiled to have more than one revolution of loop. This allows the duct 308 and the frame 306 to form a closed loop that is smaller in profile than a similar sized dispenser and duct without C-channels. This in turn can reduce the packaging size and storage space for the assembly shown. Where the end clip 434 is instead an intermediate clip 430 with two pass-through slots 426, the second end 308b of the tubing may be capped to cover the open second end.

The guidewire 302 can have a distal end that is located at the tip of the first branch 312 and can optionally project into the adaptor in the assembly state shown. The guidewire can extend into the duct or tubing 308 and be coiled with the tubing. The second end of the guidewire 302 can extend up to the second end 308b of the duct but can terminate short of the second end 308b, which depends on the overall guidewire length desired for the particular catheter assembly.

Similar to previously discussed guidewires, the guidewire 302 usable with the guidewire dispenser 292 can include at least two distinct guidewire sections with two different guidewire diameters. The smaller diameter distal guidewire section can pass through the needle tip and into the vein to guide the catheter tube while a shoulder, gradual taper, and/or larger second diameter proximal section can be stopped at the crimp section of the needle to delimit further distal travel of the guidewire.

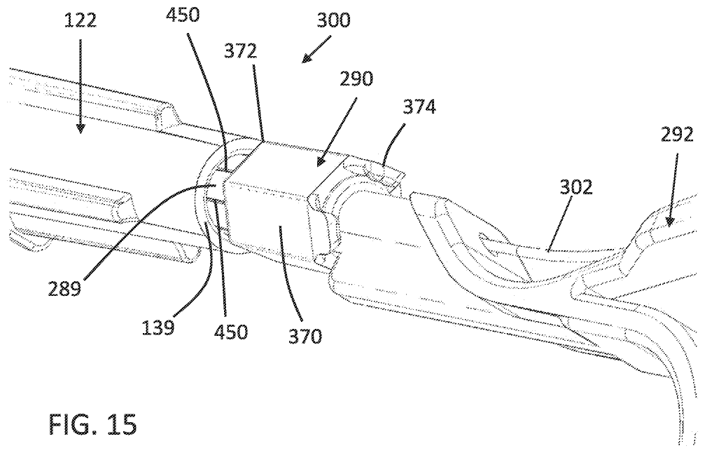

With reference now to FIG. 15, a partial perspective view of a piercing and guiding unit 300 or PG unit in accordance with further aspects of the invention is shown. Like other PG units discussed elsewhere herein, the PG unit 300 of the present embodiment comprises a needle hub 122 with a needle (not shown), an adaptor 290, and a guidewire dispenser 292 with a guidewire 302. The components of the present PG unit 300 can be similar to components discussed elsewhere with a few differences, as further discussed below. Further, the needle hub 122 with needle may be omitted to convert the present PG unit to a guiding unit, similar to guiding unit of FIG. 14, that can include a protective cap located over the tip of the adaptor 290, which can then be packaged and shipped as a combination guiding unit.

In the present embodiment, a plurality of gas channels 450 are incorporated with the adaptor 290. As shown, the plurality of gas channels 450 are located on the tip portion of the adaptor, distal of the base 370. The plurality of gas channels 450 can each be axially arranged, relative to the longitudinal axis of the adaptor 290. The plurality of gas channels 450 can be spaced from one another. In a preferred embodiment, the plurality of gas channels 450 are evenly spaced around the exterior of the tip of the adaptor. Most preferably, the gas channels 450 are exposed to the atmosphere at a gap 289 between the proximal end edge 139 of the needle hub 122 and the shoulder 372 on the adaptor 290.

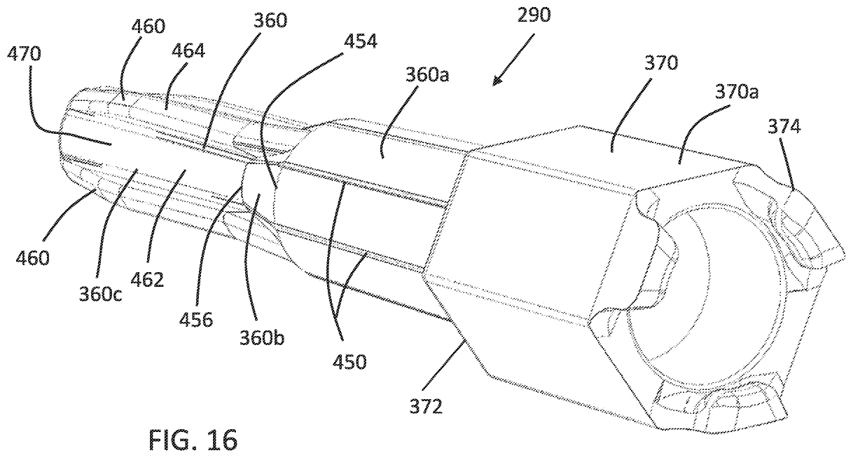

FIG. 16 is a rear perspective view of the adaptor 290 of FIG. 15, separated from the other components. Like other adaptors discussed elsewhere, the present adaptor 290 comprises a base 370, a tip 360, and a shoulder 372 located therebetween. In the present embodiment, the tip 360 has a first tip section 360a, a second tip section 360b, and a third tip section 360c. The second tip section 360b can be a reducer or a transition section to transition the outer diameter size of the first tip section 360a down to the outer diameter size of the third tip section 360c. The second tip section 360b has a first end 454 that connects or joins to the first tip section 360a and a second end 456 that connects or joins to the third tip section 360c, and wherein the first end 454 of the transition section has a larger outer diameter than the second end 456.

In an example, the first tip section 360a is generally cylindrical. In some examples, the first tip section has a taper or a draft angle in the proximal to distal direction. The third section 360c can similarly be generally cylindrical. In some examples, the third tip section has a taper or a draft angle from the proximal to distal direction.

A plurality of elongated ribs 460 are provided on the first and second tip sections 360b, 360c. Each rib 460 has a length extending in the axial direction of the adaptor, a width measured orthogonally to the length, and height measured from the exterior surface 462 of the tip to the top surface 464 of the rib. In an example, the collective top surfaces 464 of the plurality of ribs 460 define an effective diameter. In some examples, the height of each rib can vary from the distal end of the tip to the second end 456 of the transition section. Thus, the effective diameter of the tip at the third tip section 360c can vary due to the variable heights of the ribs. When incorporated, the effective diameter of the tip at the third tip section 360c can create a taper, or no taper, to enable the tip 360 to fit inside the bore of the needle hub and match the taper contour of the bore of the needle hub. The effective diameter of the tip at the second tip section 360b can similarly be selected to fit inside the bore of the needle hub and match the taper contour of the bore.

Preferably, the effective diameter defined by the plurality of ribs and the exterior of the first tip section 360a form a continuous outer diameter profile that can fittingly couple to a bore of a needle hub when inserting the tip into the proximal opening of the needle hub. The effective diameter at the distal end of the tip 360 is preferably smaller than the rest of the effective diameter so as to create a reduced tip end to facilitate insertion of the tip into the needle hub.

A ravine 470 is defined between two adjacent elongated ribs 460. Each ravine 470 has a width and a length. Relative to the effective diameter of the tip, each ravine also has a depth measured relative to the top surfaces 464 of the adjacent ribs 460. Thus, the tip 360 of the present embodiment comprises a plurality of ribs 460 and a plurality of ravines 470. In an example, four elongated ribs 460 defining four ravines 470 are provided on the tip of the present adaptor 290. In some examples, there can be fewer than four ribs or more than four ribs, such as three ribs, five ribs, or six ribs, defining three ravines, five ravines, or six ravines, respectively.

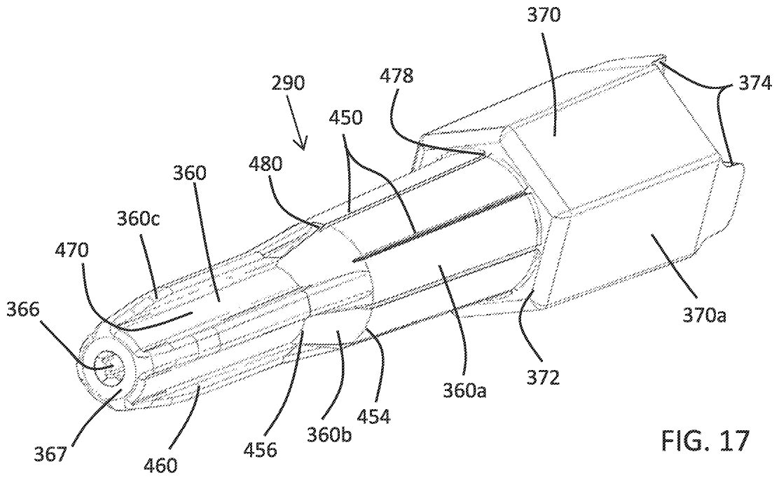

With reference to FIG. 17 and further reference to FIG. 16, the ribs 460 are preferably spaced apart around the periphery of the tip 360 to define four generally same sized ravines 470. However, the tip of the present adaptor can still be inserted into the proximal opening of a needle hub and couple to the needle hub when unequally spaced ribs and ravines of different sizes are incorporated. Also shown in both FIGS. 16 and 17 are a plurality of gas channels 450. As further discussed below, the gas channels are sized and shaped to permit gas, such as air, to travel inside the channels to escape from the interior space of the needle hub but not large enough to permit viscous fluids, such as blood, to freely flow therein.

FIG. 17 shows a front perspective view of the adaptor 290 of FIG. 16, showing several of the ribs 460, ravines 470, and distal opening 366 at the distal end 367. FIG. 17 also clearly shows the plurality of gas channels 450. In the example shown, each gas channel 450 is aligned axially relative to the longitudinal axis of the adaptor 290. Each gas channel can have a first channel end 478 located at about the shoulder 372 and a second channel end 480 located distal of the first end 454 of the second tip section 360b. In an example, the second channel end 480 can originate or terminate, depending on the viewing perspective, somewhere on the third tip section 360c.

The second channel end 480 of each gas channel 450 is preferably located somewhere between the first end 454 and the second end 456 of the second tip section 360b. The first and second channel ends 478, 480 are understood to form two ends of each gas channel 450. Less preferably, different second channel ends 480 of a plurality of gas channels 450 are located along different sections of the tip 360, such as some being located at the second tip section 360b while others are located at the third tip section 360c.

As discussed above, the first channel end 478 of each gas channel 374 can originate or terminate at the shoulder 372 between the tip 360 and the base 370. In some examples, the first channel ends 478 of some or all of the gas channels 374 can be located distal of the shoulder 372, somewhere along the first tip section 360a at the gap 289 when the adaptor 290 is coupled to the needle hub 122 and the guidewire dispenser 292 (FIGS. 8 and 15).

The gas channels 450 allow air to escape by entering through the second channel end 480 and flowing in the proximal direction in the respective gas channel 450. Without the gas channels, the first tip section 360a can form a continuous taper fit with the interior of the needle hub. Thus, air trapped inside needle hub during blood flashback may not have sufficient vent openings in the system for venting. The gas channels 450 incorporated with the present adaptor provides air passages through an otherwise continuous seal between the interior of the needle hub and the exterior of the tip 360, such as the exterior of the first tip section 360a.

When the present adaptor 290 with the gas channels is used as part of a PG unit with a catheter assembly, initial puncture of a vein will cause blood to enter the needle lumen and into the interior space of the needle hub, known as primary blood flashback. By incorporating the plurality of gas channels 450, air can be exhausted out of the needle hub interior space as blood travels into the flashback chamber of the needle hub, thereby promoting blood flashback flow and minimizing vapor lock when no air or little air is allowed to escape. In an example, four or more gas channels 450 can be incorporated with the tip 360. Preferably, six evenly spaced gas channels 450 are incorporated with the tip 360. More preferably, eight gas channels 450 are incorporated with the tip 360. However, more than eight gas channels 450 can be incorporated to increase the available channels for venting.

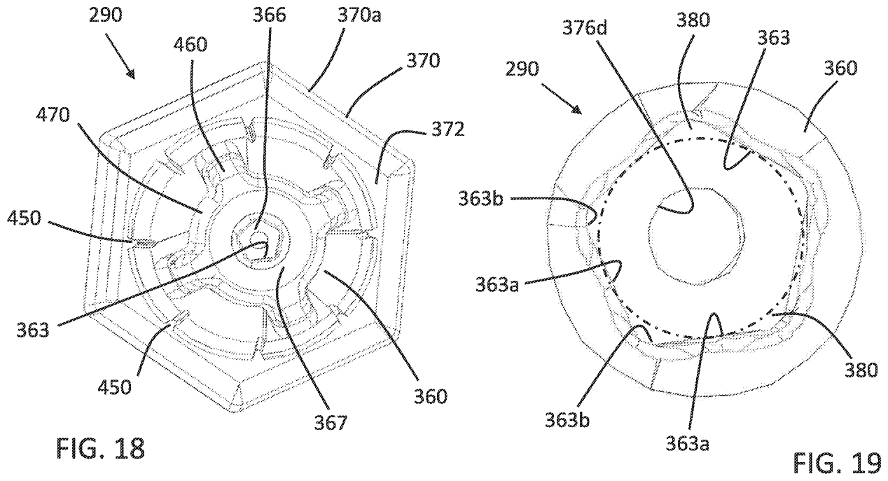

With reference now to FIG. 18, an end view of the adaptor 290 of FIG. 17 is shown, looking at the distal end 367 and the distal opening 366 of the tip 360. The end view of FIG. 18 clearly shows four elongated ribs 460, four ravines 470, and eight gas channels 450 at the tip 360 of the adaptor 290. As previously discussed, the number of ribs, ravines, and gas channels can vary with the numbers of each can be a variable that can be selected to ensure adequate blood flashback and air flow or exhaust during vascular access.

As shown in FIG. 18, each gas channel 450 has two sidewalls and a bottom wall, forming a generally U-shaped surface feature on the tip 360 of the adaptor 290. The length and width of each U-shaped surface should be selected to permit gas or air flow but not the free flowing of blood. The cross-sectional shape of the gas channel can also vary, such as being generally round, generally C-shaped, etc.

Wall surfaces of the cannula passage 363 of the adaptor 290 is shown in FIG. 18 through the distal opening 366 at the tip 360. The cannula passage 363 of the present adaptor 290 is similar to the cannula passage 363 of the adaptor 290 of FIG. 6A. The cannula passage 363 is also shown in the enlarged view of FIG. 19. As shown, the cannula channel 363 has a non-circular interior space defined by five generally straight sides 363a connected to one another by arcuate connecting sections 363b. Thus, the present embodiment has five generally straight sides 363a connected to one another by five arcuate connecting sections 363b to define the non-circular cannula channel or passage 363.

If the proximal end of a needle is placed into the cannula passage 363 of the present embodiment, the needle proximal end would resemble the ring-shaped structure shown in dashed lines in FIG. 19 and would contact, be in close contact, or has some play with the five generally straight sides 363a of the cannula channel 363. The needle bore would align centrally with the guidewire passage 376d shown. The contact between the exterior of the needle and the five generally straight sides 363a can generate a slight interference fit for a firm connection between the cannula channel 363 and the needle. However, interference fit is not required, and some play can be practiced to limit physical contact between the needle and the relatively softer material of the adaptor. Additionally, the play can facilitate assembly without perfect concentricity. Each arcuate connection section 363b and the needle exterior define a bypass gap 380 at the cannula channel 363.

As shown, the tip 360 of the adaptor 290 and the dashed lines representing a needle at the cannula channel 363 define five bypass gaps 380. In other examples, there can be more than five straight sides and corresponding number of arcuate connection sections or less than five, such as four. In still other examples, the cannula channel 363 contour can have different shapes than as shown. For example, the five straight lines, where they contact the needle, can be slightly arcuate, with a different curvature than the arc of the needle outer diameter. In still yet other examples, the present cannula channel 363 can be similar to the cannula channel of FIG. 6C.

As previously described with reference to FIG. 6C, because the bypass gaps 380 can be sized for blood flow, such as by providing a sufficient flow passage or clearance at each bypass for a given fluid viscosity, blood entering the cannula channel 363 can flow out through the bypass gaps 380 and into the interior space (362 of FIG. 4B) of the needle hub. A practitioner can thus detect primary blood flashback at the interior space of the needle hub after blood flashback flows into the cannula channel 363 of the adaptor 290 and then reverses flow and flow out through the bypass gaps 380 and into the interior space of the needle hub to be detected visually through the wall surfaces of the needle hub, such as by perceiving the blood red color within the interior space.

As can be understood from the immediate prior paragraphs, the bore of the present adapter 290 can have a plurality of bore sections, a guidewire passage, and a cannula channel, similar to the adaptor of FIG. 6A. In some examples, the bore of the present adaptor can incorporate a valve chamber for accommodating a valve, similar to the bore of the adaptor of FIG. 9 for accommodating one of the valves described elsewhere herein, such as the valves of FIGS. 10 and 11. In still yet another example, the tip of the present adaptor is spaced from the cannula proximal end, similar to that shown in FIGS. 12A and 12B.

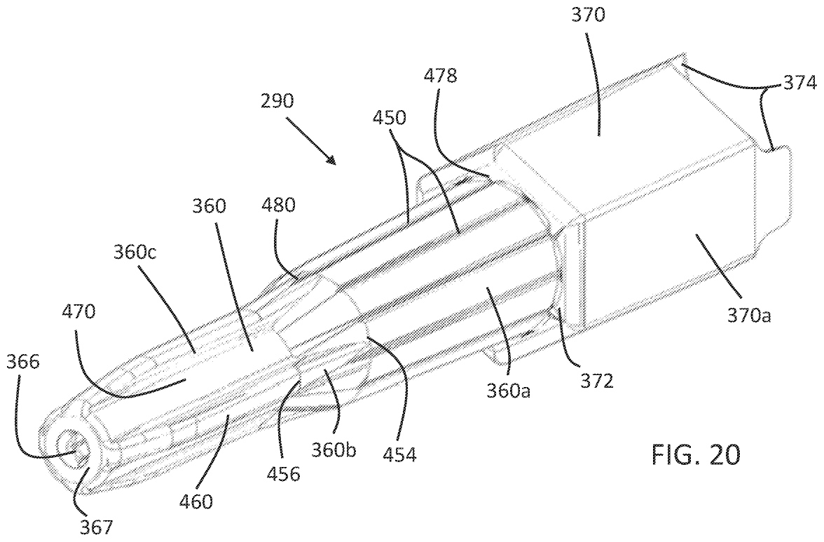

FIG. 20 is a front perspective view of an adaptor 290 that is similar to the adaptor 290 of FIGS. 16-19, with a few changes. In the present adaptor 290, the gas channels 450 are more closely spaced together, relative to the gas channels of FIGS. 16-19. In an example, additional gas channels 450 are incorporated to more closely spaced together the gas channels 450.

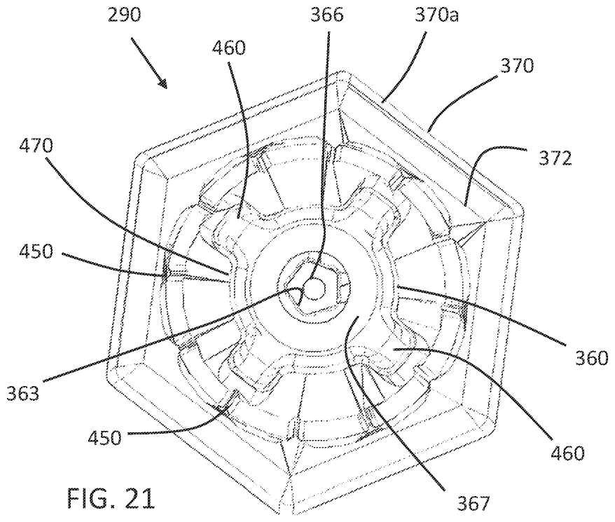

With further reference to FIG. 21, which shows a front-end view of the adaptor of FIG. 20, twelve gas channels 450 are incorporated to increase the number of channels for venting. However, and as previously discussed, more than or less than twelve gas channels can be incorporated. For example, at least three gas channels 450, at least four gas channels, at least eight gas channels, and twelve gas channels can be incorporated on the tip for venting. In an example, four to twelve gas channels 450 can be incorporated. In the example shown, the twelve gas channels 450 are generally evenly spaced about the first tip section 360a. However, the spacing between the gas channels 450 can be unevenly spaced to direct more venting towards one are of the tip than others.

In an example, four of the gas channels 450 are axially aligned with the four elongated ribs 460on on the first and second tip sections 360b, 360c. The four aligned gas channels 450 with the four elongated ribs 460 permit air to vent at locations where the four ribs 460 are in close spacing with the interior of the needle hub, when the adaptor 290 is connected to the needle hub. However, the alignment is not required, and air can still vent by incorporating an appropriate number of gas channels 450, such as six to fourteen, with eight to twelve being more preferred.

Methods of making and of using catheter assemblies and components thereof and components for using with catheter assemblies are within the scope of the present invention.

The present disclosure is also described by the following embodiments:

Embodiment 1: A catheter assembly comprising:
a catheter tube attached to a catheter hub and a needle attached to a needle hub, wherein the needle is located in a lumen of the catheter tube and a needle tip of the needle projects out a distal opening of the catheter tube;
an adaptor having a body comprising a base, a tip, and a bore extending through the body, the base having a base bore section and the tip having tip bore section with a frustoconical shape, the tip located in a proximal opening of the needle hub; and
a guidewire dispenser having a frame with a plurality of branches, a connection tip at a first branch of the plurality of branches located inside the base bore section of the adaptor; and
wherein a guidewire extends through a second branch of the plurality of branches and in through the connection tip and into the bore of the adaptor.

Embodiment 2: The catheter assembly of embodiment 1, further comprising a housing comprising a first frame attached to a second frame and having an interior space, said housing having a distal opening and a proximal opening and the catheter hub is located at least in part in the interior space of the housing.

Embodiment 3: The catheter assembly of embodiment 1, further comprising a duct having a first end attached to a socket at the second branch and wherein the guidewire extends at least partially into the duct.

Embodiment 4: The catheter assembly of embodiment 3, wherein the duct has a second end attached to a socket of a third branch of the plurality of branches.

Embodiment 5: The catheter assembly of embodiment 4, wherein the duct and the frame define an enclosed loop.

Embodiment 6: The catheter assembly of embodiment 1, further comprising a valve located in the bore of the adaptor.

Embodiment 7: The catheter assembly of embodiment 6, wherein the valve comprises a valve disc and a skirt extending therefrom.

Embodiment 8: The catheter assembly of embodiment 1, further comprising a needle guard located in an interior of the catheter hub.

Embodiment 9: The catheter assembly of embodiment 1, wherein the needle has a crimp and wherein the guidewire abuts the needle at the crimp to delimit distal travel of the guidewire.

Embodiment 10: The catheter assembly of embodiment 1, wherein the guidewire has at least two guidewire sections with at least two different diameters.

Embodiment 11: The catheter assembly of embodiment 10, wherein the guidewire has a distal guidewire section of a first diameter and a proximal guidewire section of a second diameter, and wherein the second diameter is larger than the first diameter.

Embodiment 12: The catheter assembly of embodiment 11, wherein the guidewire comprises a shoulder or a gradual taper located at a point between the distal guidewire section and the proximal guidewire section.

Embodiment 13: The catheter assembly of embodiment 12, wherein at least one of the shoulders, the gradual taper, and the second diameter is engageable with the needle at a crimp of the needle.

Embodiment 14: The catheter assembly of embodiment 13, wherein a location of the shoulder or the gradual tapers defines a length of the distal guidewire section that extends out the needle tip when the guidewire engages the needle at the crimp.

Embodiment 15: The catheter assembly of embodiment 12, wherein the distal guidewire section passes through the needle at the crimp and extends out an opening at the needle tip.

Embodiment 16: The catheter assembly of embodiment 12, wherein the guidewire has a marker or an indicator to indicate how far the distal guidewire section has extended distally beyond the needle tip.

Embodiment 17: The catheter assembly of embodiment 16, wherein the marker or indicator indicates at least one of when a first end of the distal guidewire section reaches the needle tip, passes through the needle tip, and advances fully out of the distal tip.

Embodiment 18: The catheter assembly of embodiment 16, wherein the guidewire has a plurality of markers or guidewires to indicate when a first end of the distal guidewire section reaches the needle tip, passes through the needle tip, and advances fully out of the distal tip.

Embodiment 19: The catheter assembly of embodiment 1, wherein the guidewire has a code, the guidewire dispenser has a code, or both the guidewire and the guidewire dispenser have codes for indicating a gauge size.

Embodiment 20: The catheter assembly of embodiment 19, wherein the code or codes include a color, an alpha code, a numeric code, an alpha numeric code, or combinations thereof.

Embodiment 21: The catheter assembly of embodiment 1, wherein the tip of the adaptor comprises a cannula channel having an inside diameter and a guidewire passage having an inside diameter, and wherein the diameter of the cannula channel is larger than the diameter of the guidewire passage.

Embodiment 22: The catheter assembly of embodiment 21, wherein the cannula channel is located distal of the guidewire passage.

Embodiment 23: The catheter assembly of embodiment 1, wherein the base of the adaptor has a body with an exterior having plurality of slats.

Embodiment 24: The catheter assembly of embodiment 23, further comprising two or more flanges extending from two or more of the slats.

Embodiment 25: The catheter assembly of embodiment 24, further comprising one of a male detent and a female detent formed with each of the two or more flanges.

Embodiment 26: The catheter assembly of embodiment 1, wherein the body at the tip of the adaptor has a cannula channel having an interior surface defining a non-circular cross-sectional opening.

Embodiment 27: The catheter assembly of embodiment 26, wherein a proximal end of the needle contacts the interior surface at the cannula channel.

Embodiment 28: The catheter assembly of embodiment 27, wherein the non-circular cross-sectional opening has a plurality of straight sides and a plurality of arcuate connecting sections.

Embodiment 29: The catheter assembly of embodiment 28, wherein bypass gaps are formed between the plurality of arcuate connecting sections and the needle.

Embodiment 30: The catheter assembly of embodiment 26, wherein the body at the tip of the adaptor has a plurality of bore sections of different internal diameters.

Embodiment 31: The catheter assembly of embodiment 1, wherein the plurality of branches comprises at least three branches, which include a first branch, a second branch, and a third branch.

Embodiment 32: The catheter assembly of embodiment 31, further comprising a tubing having a first end, a second end, and a lumen and wherein the guidewire is disposed at least in part in the lumen of the tubing.

Embodiment 33: The catheter assembly of embodiment 32, wherein the first end of the tubing is located in a socket at the second branch and the second end of the tubing is either located in a socket at the third branch or is remote from the third branch.

Embodiment 34: The catheter assembly of embodiment 33, further comprising an intermediate retaining clip gripping different sections of the tubing.

Embodiment 35: The catheter assembly of embodiment 1, wherein a valve is located in the catheter hub.

Embodiment 36: A catheter assembly comprising:
a catheter tube attached to a catheter hub and a needle having a needle tip attached to a needle hub, wherein the needle projects through the catheter tube and the needle tip disposed distally of a distal opening of the catheter tube;
an adaptor having a body with an exterior surface and an interior surface defining a bore, the body further having a base with a plurality of slats connected to one another along respective edges and a tip having a frustoconical section having a guidewire passage and a cannula channel distal of the guidewire passage, said tip located in a proximal opening of the needle hub;
a guidewire dispenser having a tip disposed in the bore of the adaptor and a guidewire having a first end disposed in the guidewire passage; and
wherein a proximal end of the needle is disposed in the cannula channel of the adaptor.

Embodiment 37: A catheter assembly comprising:
a catheter tube attached to a catheter hub and a needle having a needle tip attached to a needle hub, wherein the needle projects through the catheter tube and the needle tip disposed distally of a distal opening of the catheter tube;
an adaptor having a body with an exterior surface and an interior surface defining a bore, the body further having a base having a base bore having a valve chamber and a valve comprising at least one slit and two flaps located in the valve chamber and a tip having a frustoconical section having a guidewire passage, said tip located in a proximal opening of the needle hub; and
a guidewire dispenser having a tip disposed in the bore of the adaptor and a guidewire having a first end disposed in the bore of the adaptor.

Embodiment 38: A method for assembly a catheter assembly comprising:
providing a catheter hub with a catheter tube extending out a distal end of the catheter hub, said catheter hub having a body with an interior cavity and said catheter tube comprising a lumen and a distal opening;
providing a needle hub with a needle having a shaft with a lumen and a distal tip, said needle hub comprising a body with an interior cavity and said shaft projecting through the lumen of the catheter tube with said distal tip projecting distally of the distal opening of the catheter tube;
providing an adaptor having a body with an exterior surface and an interior surface defining a bore, the body further having a base having a base bore and a tip having a frustoconical section having a guidewire passage, said tip of said adaptor located in the interior cavity at a proximal opening of the needle hub; and
providing a guidewire dispenser, said guidewire dispenser having a tip disposed in the base bore of the adaptor and a guidewire having a first end disposed in the base bore of the adaptor.

Embodiment 39: A method of using a catheter assembly with a guidewire comprising:
puncturing a vein with a needle tip of a needle, which has a needle crimp;
advancing a guidewire through an adaptor connected to a needle hub having the needle attached thereto; and
further advancing the guidewire so that a distal guidewire section extends out the needle tip and the guidewire engages the needle at needle crimp.

Embodiment 40: The method of embodiment 39, wherein the guidewire is advanced without a guidewire dispenser.

Embodiment 41: The method of embodiment 40, wherein the guidewire is at least partially located in a guidewire dispenser and wherein the guidewire dispenser has a tip connected to a base of the adaptor.

Embodiment 42: An adaptor for use to guide a guidewire through a needle lumen comprising:
a body comprising a base, a tip, and a bore extending through the body; the base having a base bore section and the tip having tip bore section with a frustoconical shape;
at least one of a cannula channel at the tip having a plurality of generally straight sides with two adjacent plurality of straight sides connected to one another by an arcuate connecting portion and sized to receive a needle shaft and a valve comprising a wall having at least one slit positioned in the base bore of the base;
wherein the base comprises a proximal opening sized and shaped for receiving a male tip of a guidewire dispenser and plurality of first engagement structures for engaging a corresponding plurality of second engagement structures on the male tip of the guidewire dispenser.

Embodiment 43: The adaptor of embodiment 42, wherein the base has a non-circular cross-section having a plurality of slats or sidewalls.

Embodiment 44: The adaptor of embodiment 43, wherein each one of the plurality of first engagement structures extends from one of the plurality of slats or sidewalls.

Embodiment 45: The adaptor of embodiment 42, wherein the cannula channel has a first inside cross-sectional dimension and a guidewire passage having a second inside cross-sectional dimension located proximally of the cannula channel; and wherein the first inside cross-sectional dimension is greater than the second inside cross-sectional dimension.

Embodiment 46: The adaptor of embodiment 42, wherein the cannula channel has three generally straight sides and three arcuate connecting portions.

Embodiment 47: The adaptor of embodiment 42, wherein the valve has a skirt extending from the wall.

Embodiment 48: The adaptor of embodiment 47, wherein the wall of the valve has a first portion with a first thickness and a second portion with a second thickness and wherein the first thickness is greater than the second thickness, measured orthogonal to a medial plane passing through the wall.

Embodiment 49: The adaptor of embodiment 42, wherein the valve is located between two internal shoulders of the base.

Embodiment 50: A guidewire dispenser comprising:
a frame with a plurality of branches, including a first branch and a second branch;
a connection tip at the first branch, said connection tip comprising a lumen and a tapered nose section;
a guide bracket at the second branch and a lumen formed through guide bracket, said guide bracket comprising a socket;
a tubing having a first free end and a second free end, said first free end located in the socket and said second free end held by a retaining clip or by socket located on a third branch;
a guidewire having a length with a first free end and a second free end, said guidewire located in said tubing and extending through the socket at the second branch and into the lumen of the connection tip; and
wherein the guidewire is exposed between the first branch and the second branch.

Embodiment 51: The guidewire dispenser of embodiment 50, wherein the third branch has an enclosed wall at an end of the socket.

Embodiment 52: The guidewire dispenser of embodiment 50, wherein the second branch is located higher, elevation-wise, than the first branch.

Embodiment 53: The guidewire dispenser of embodiment 50, wherein the tubing and the frame form an enclosed loop.

Embodiment 54: The guidewire dispenser of embodiment 50, wherein the guidewire has a first section with a first diameter and a second section with a second diameter larger than the first diameter.

Embodiment 55: The guidewire dispenser of embodiment 50, wherein the frame has a Y-shape configuration.

Embodiment 56: The guidewire dispenser of embodiment 55, wherein the retaining clip is a first retaining clip and further comprising a second retaining clip having two pass-through slots.

Although limited embodiments of the extended dwell catheter assemblies and their components have been specifically described and illustrated herein, many modifications and variations will be apparent to those skilled in the art. For example, the locations and placements of the male and female detents can be reversed at the joining edges, the gripping surfaces can be non-symmetrical, and the materials can be modified, such as with insert molding or co-molding. Accordingly, it is to be understood that the extended dwell catheter assemblies and their components constructed according to principles of the disclosed device, system, and method may be embodied other than as specifically described herein. The disclosure is also defined in the following claims.

1-67. (canceled)

68. A catheter assembly comprising:
a catheter tube attached to a catheter hub and a needle attached to a needle hub, wherein the needle is located in a lumen of the catheter tube and a needle tip of the needle projects out a distal opening of the catheter tube;
an adaptor having a body comprising a base, a tip, and a bore extending through the body, the base having a base bore section and the tip having a tip bore section, the tip located in a proximal opening of the needle hub; and
a guidewire dispenser having a frame with a plurality of branches, a connection tip at a first branch of the plurality of branches located inside the base bore section of the adaptor; and
wherein a guidewire extends through a second branch of the plurality of branches and in through the connection tip at the first branch.

69. The catheter assembly of claim 68, further comprising a housing comprising a first frame attached to a second frame and having an interior space, said housing having a distal opening and a proximal opening and the catheter hub is located at least in part in the interior space of the housing.

70. The catheter assembly of claim 68, further comprising a duct having a first end (308a) attached to a socket at the second branch and wherein the guidewire extends at least partially into the duct.

71. The catheter assembly of claim 70, wherein the duct has a second end attached to a socket of a third branch of the plurality of branches.

72. The catheter assembly of claim 68, further comprising a valve located in the bore of the adaptor.

73. The catheter assembly of claim 68, further comprising a needle guard located in an interior cavity of the catheter hub.

74. The catheter assembly of claim 68, wherein the guidewire has at least two guidewire sections with at least two different diameters.

75. The catheter assembly of claim 68, wherein the tip of the adaptor comprises a plurality of spaced apart gas channels.

76. The catheter assembly of claim 75, further comprising a plurality of spaced apart elongated ribs located on an exterior of the tip, and wherein a ravine comprising a width and a length is defined between two adjacent elongated ribs.

77. The catheter assembly of claim 74, wherein the guidewire comprises a shoulder or a gradual taper located at a point between the distal guidewire section and the proximal guidewire section.

78. The catheter assembly of claim 77, wherein the distal guidewire section passes through the needle at the crimp and extends out an opening at the needle tip.

79. The catheter assembly of claim 68, wherein the tip of the adaptor comprises a cannula channel having an inside measurement and a guidewire passage having an inside measurement, and wherein the inside measurement of the cannula channel is larger than the inside measurement of the guidewire passage.

80. The catheter assembly of claim 79, wherein the cannula channel is located distal of the guidewire passage.

81. The catheter assembly of claim 68, wherein the base of the adaptor is formed with a plurality of slats.

82. The catheter assembly of claim 68, wherein the body at the tip of the adaptor has a cannula channel having an interior surface defining a non-circular cross-sectional opening.

83. The catheter assembly of claim 68, wherein the plurality of branches comprises at least three branches, which include a first branch, a second branch, and a third branch.

84. The catheter assembly of claim 83, further comprising a tubing having a first end, a second end, and a lumen and wherein the guidewire is disposed at least in part in the lumen of the tubing.

85. The catheter assembly of claim 75, wherein the plurality of gas channels comprises four to twelve gas channels.

86. A catheter assembly comprising:
a catheter tube attached to a catheter hub and a needle having a needle tip attached to a needle hub, wherein the needle projects through the catheter tube and the needle tip disposed distally of a distal opening of the catheter tube;
an adaptor having a body with an exterior surface and an interior surface defining a bore, the body further having a base having a base bore having a valve chamber and a valve comprising at least one slit and two flaps located in the valve chamber and a tip having a guidewire passage, said tip located in a proximal opening of the needle hub; and
a guidewire dispenser having a tip disposed in the bore of the adaptor and a guidewire having a first end disposed in the bore of the adaptor.

87. The catheter assembly of claim 86, further comprising a plurality of spaced apart gas channels disposed on the exterior surface of the body of the adaptor.

88. The catheter assembly of claim 86, further comprising a plurality elongated ribs disposed on the exterior surface of the body of the adaptor.

89. An adaptor for use to guide a guidewire through a needle lumen comprising:
a body comprising a base, a tip, and a bore extending through the body; the base having a base bore section and the tip having a tip bore section;
at least one of a cannula channel at the tip having a plurality of generally straight sides with two adjacent plurality of straight sides connected to one another by an arcuate connecting portion and sized to receive a needle shaft and a valve comprising a wall having at least one slit positioned in the base bore of the base;
wherein the base comprises a proximal opening sized and shaped for receiving a tip of a guidewire dispenser and a first engagement structure for engaging a second engagement structure on the tip of the guidewire dispenser.

90. The adaptor of claim 89, wherein the base has a non-circular cross-section having a plurality of slats or sidewalls.

91. The adaptor of claim 90, wherein the first engagement structure extends from one of the plurality of slats or sidewalls.

92. The adaptor of claim 89, wherein the cannula channel has a first inside cross-sectional dimension and a guidewire passage having a second inside cross-sectional dimension located proximally of the cannula channel; and wherein the first inside cross-sectional dimension is greater than the second inside cross-sectional dimension.

93. The adaptor of claim 89, wherein the cannula channel has a plurality of generally straight sides and wherein two adjacent generally straight sides are connected by an arcuate connecting portion.

94. The adaptor of claim 89, wherein the valve has a skirt extending from the wall.

95. The adaptor of claim 94, wherein the wall of the valve has a first portion with a first thickness and a second portion with a second thickness and wherein the first thickness is greater than the second thickness, measured orthogonal to a medial plane passing through the wall.

96. The adaptor of claim 89, wherein the valve is located between two internal shoulders of the base.

97. The adaptor of claim 89, wherein the tip having the tip bore section with a frustoconical shape.

98. A guidewire dispenser comprising:
a frame with a plurality of branches, including a first branch and a second branch;
a connection tip at the first branch, said connection tip comprising a lumen;
a guide bracket at the second branch and a lumen formed through guide bracket, said guide bracket comprising a socket;
a tubing having a first free end and a second free end, said first free end located in the socket and said second free end held by a retaining clip or by socket located on a third branch;
a guidewire having a length with a first free end and a second free end, said guidewire located in said tubing and extending through the socket at the second branch and into the lumen of the connection tip; and
wherein the guidewire is exposed between the first branch and the second branch.

99. The guidewire dispenser of claim 98, wherein the third branch has an enclosed wall at an end of the socket.

100. The guidewire dispenser of claim 98, wherein the second branch is located higher, elevation-wise, than the first branch.

101. The guidewire dispenser of claim 96, wherein the tubing and the frame form a closed loop.

102. The guidewire dispenser of any claim 98, wherein the frame has a Y-shape configuration.
